(19) United States
(12) Patent Application Publication
(10) Pub. No.: US 2023/0338427 A1
(43) Pub. Date: Oct. 26, 2023

(54) MITOCHONDRIAL AUGMENTATION THERAPY

(71) Applicant: Minovia Therapeutics Ltd., Tirat Hacarmel (IL)

(72) Inventors: Natalie Yivgi-Ohana, Haifa (IL); Noa Sher, Haifa (IL); Moriya Blumkin, Tel Aviv (IL)

(21) Appl. No.: 17/912,022

(22) PCT Filed: Mar. 29, 2021
(86) PCT No.: PCT/IL2021/050349

§ 371 Date: Sep. 15, 2022

Related U.S. Application Data
(60) Provisional application No. 63/118,569, filed on Nov. 25, 2020.
(60) Provisional application No. 63/003,174, filed on Mar. 31, 2020.

(51) Int. Cl. A61K 35/28; A61P 7/00; A61K 35/545; A61P 37/06

(52) U.S. Cl. CPC A61K 35/28; A61P 7/00; A61K 35/545; A61P 37/06

(57) ABSTRACT

The present invention provides methods and compositions which cause the bone marrow to increase production of leukocyte cells. Specifically, the invention provides methods and compositions for increasing levels of CD45+ cells in a subject by providing mitochondrially-enriched cells. Further the present application provides methods for increasing bone marrow cellularity, engraftment of CD34+ cells and differentiation.

### CROSS-REFERENCE TO RELATED APPLICATIONS

This application claims benefit of priority under 35 U.S.C. § 119(e) of U.S. Ser. No. 63/118,569, filed on Nov. 25, 2020, and U.S. Ser. No. 63/003,174, filed on Mar. 31, 2020, the entire contents of both are incorporated herein by reference in its entirety.

### BACKGROUND OF THE INVENTION

### Field of the Invention

The present invention relates generally to cells enriched with mitochondria and more specifically to methods and compositions for increasing stem cell engraftment, proliferation, homing or survival, and modifying stem cell differentiation patterns.

### Background Information

The mitochondrion is a membrane bound organelle found in most eukaryotic cells, ranging from 0.5 to 1.0 pm in diameter. Mitochondria are found in nearly all eukaryotic cells and vary in number and location depending on the cell type. Mitochondria contain their own DNA (mtDNA) and their own machinery for synthesizing RNA and proteins. The mtDNA contains only 37 genes, thus most of the gene products in the mammalian body are encoded by nuclear DNA.

Mitochondria perform numerous essential tasks in the eukaryotic cell such as pyruvate oxidation, the Krebs cycle and metabolism of amino acids, fatty acids and steroids. However, the primary function of mitochondria is the generation of energy as adenosine triphosphate (ATP) by means of the electron-transport chain and the oxidative-phosphorylation system (the “respiratory chain”). Additional processes in which mitochondria are involved include heat production, storage of calcium ions, calcium signaling, programmed cell death (apoptosis) and cellular proliferation.

The ATP concentration inside the cell is typically 1-10 mM ATP can be produced by redox reactions using simple and complex sugars (carbohydrates) or lipids as an energy source. For complex fuels to be synthesized into ATP, they first need to be broken down into smaller, simpler molecules. Complex carbohydrates are hydrolyzed into simple sugars, such as glucose and fructose. Fats (triglycerides) are metabolized to give fatty acids and glycerol.

The overall process of oxidizing glucose to carbon dioxide is known as cellular respiration and can produce about 30 molecules of ATP from a single molecule of glucose. ATP can be produced by a number of distinct cellular processes. The three main pathways used to generate energy in eukaryotic organisms are glycolysis and the citric acid cycle/oxidative phosphorylation, both components of cellular respiration, and beta-oxidation.

The majority of this ATP production by non-photosynthetic eukaryotes takes place in the mitochondria, which can make up nearly 25% of the total volume of a typical cell. Various mitochondrial disorders are known to result from defective genes in the mitochondrial DNA.

Leukocytes are the cells of the immune system that are involved in protecting the body against both infectious disease and foreign invaders. Leukocytes comprise different types of cells which can be distinguished by their physical and functional characteristics.

The bone marrow is the primary site of new blood cell production or hematopoiesis. All types of hematopoietic cells, including leukocytes are created in bone marrow. Bone marrow failure is the primary pathological feature in different disease (e.g. Fanconi anemia). Hematopoietic stem cell (HSC) transplant therapy can be administered to a subject in need of treatment so as to populate or repopulate one or more blood cell types, such as a blood cell lineage that is deficient or defective in a patient suffering from a stem cell disorder (for example Mucopolysaccharide Storage Hurler disease). However, while HSCs have significant therapeutic potential, a limitation that has hindered their use in the clinic has been the difficulty associated with ensuring engraftment of hematopoietic stem cell transplants in a host.

A patient's own immune system often attacks the exogenous (autologous, allogeneic, or syngeneic) transplanted cells and mediates rejection of the transplanted hematopoietic stem cells.

In order to avoid rejection, a patient is treated with immune system destroying agents prior to hematopoietic stem cell transplantation, e.g., chemotherapeutic agents or radiation. Unfortunately, efforts to induce tolerance of the hematopoietic stem cell transplantation in the patient often result in serious complications. Thus, there is a need for new compositions and methods to improve hematopoietic stem cell transplantation.

Leukocytes can be divided into two main categories—myeloblasts and lymphoblasts. Lymphocytes are a subtype of a white blood cell and include natural killer cells (which function in cell-mediated, cytotoxic innate immunity), T cells (for cell-mediated, cytotoxic adaptive immunity), and B cells (for humoral, antibody-driven adaptive immunity). A myelocyte is a young cell of the granulocytic series, occurring normally in bone marrow. Myelocytes mature into neutrophils, eosinophils and basophils all of which play important roles in the immune system. Shifting the production of lymphoid and myeloid cells in the bone marrow can result in the ability to induce specific immune system responses. There are numerous disease and disorders known to be related to low lymphocyte count. Additionally, a decline in immune function and an alteration in the frequency of circulating lymphocytes was reported with advancing age.

To date, there remains a need for novel and safe methods to increase leukocyte content, to improve the function of deficient immune systems, and to treat various diseases and disorders.

### SUMMARY OF THE INVENTION

The present invention is based on the seminal discovery that cells enriched with mitochondria are useful for treating diseases and disorders. The present invention provides methods and compositions which cause the bone marrow to increase production of leukocyte cells. Specifically, the invention provides methods and compositions for increasing levels of CD45+ cells in a subject by providing mitochondrially-enriched cells. Further the present application provides methods for increasing bone marrow cellularity, engraftment of CD34+ cells and differentiation of hematopoietic stem cells.

In one embodiment, the present invention provides a method of increasing levels of leukocyte cells in a subject including obtaining target cells from a subject having a disease or disorder or a donor; obtaining exogenous mitochondria; producing mitochondrially-enriched target cells by contacting the target cells with the exogenous mitochondria under conditions allowing the exogenous mitochondria to enter the target cells; and administering the mitochondrially-enriched target cells to the subject; wherein the mitochondrial content of the mitochondrially-enriched target cells is detectably higher than the mitochondrial content of the target cells, thereby increasing the levels of leukocyte cells in a subject.

The present invention also provides methods and compositions which cause the bone marrow to increase the proportion of lymphoid cells to myeloid cells. Further, the invention provides methods and compositions for modifying the levels of CD3+ cells, CD14+ cells, CD19+ cells, CD33+ cells. The invention also provides increasing the level of CD3+ cells relative to the level of CD33+ cells. In some embodiments, the invention also provides increasing the level of CD19+.

In one embodiment, the present invention provides a method of increasing levels of leukocyte cells in a subject including obtaining target cells from a subject having a disease or disorder or from a healthy donor; obtaining exogenous mitochondria; producing mitochondrially-enriched target cells by contacting the target cells with the exogenous mitochondria under conditions allowing the exogenous mitochondria to enter the target cells; and administering the mitochondrially-enriched target cells to the subject; wherein the mitochondrial content of the mitochondrially-enriched target cells is detectably higher than the mitochondrial content of the target cells, thereby causing a shift from myeloid cells to lymphoid cells in a subject.

In one aspect, the target cells are pluripotent stem cells, embryonic stem cells, induced pluripotent stem cells, mesenchymal stem cells, hematopoietic stem cells, hematopoietic progenitor cells, common myeloid progenitor cells, common lymphoid progenitor cells, CD34+ cells or any combination thereof. In certain aspects, the target cells are CD34+ cells. In an additional aspect, the target cells are obtained from whole blood, blood fractions, peripheral blood, PBMC, serum, plasma, adipose tissue, placenta, oral mucosa, blood, umbilical cord blood or bone marrow. In an additional aspect, the target cells are from a donor. In a further aspect, the target cells and/or exogenous mitochondria are autologous.

In certain aspects the subject has a disease or disorder. In some aspects, the disease or disorder is an age related disorder, cancer, muscle diseases and disorders, glycogen-storage diseases and disorders, vascular endothelium disorder or diseases, brain disorder or brain disease, placental disorder or placental disease, thymus disorder or thymus disease, autoimmune diseases, renal disease or renal disorder, primary mitochondrial disease, pancreas disorder or pancreas disease, prostate disorder or prostate disease, kidney disorder or kidney disease, blood disorder or blood disease, heart disease or heart disorder, skin disorder or skin disease, immune and inflammatory diseases and disorders, bone disease or bone disorder, gastro-intestinal disease or gastro-intestinal disorder, eye disease or eye disorder or infections.

In a further aspect, the exogenous mitochondria are isolated frozen-thawed human mitochondria. In a further aspect, the exogenous mitochondria are derived from a human cell or a human tissue. In some embodiments, the human cell or human tissue is selected from the group consisting of placenta, placental cells grown in culture, and blood cells. In some embodiments, the human cell is a human stem cell. In some embodiments, the human cell is a human somatic cell. In certain aspects, the conditions allowing the exogenous mitochondria to enter the target cells comprise incubating the target cells with the exogenous mitochondria for a time ranging from about 0.5 to 30 hours at a temperature ranging from about 16 to 37° C. In an additional aspect, the conditions allowing the exogenous mitochondria to enter the target cells comprise incubating the target cells with the exogenous mitochondria at a ratio of about 0.088-176 mU citrate synthase (CS) activity per 10⁶ cells. In one aspect, the exogenous mitochondria constitute at least 1% of the total mitochondria content in the mitochondrially-enriched target cells.

In one aspect, the exogenous mitochondrial content of the mitochondrially-enriched target cells is determined by an assay selected from the group consisting of: content of at least one mitochondrial protein selected from SDHA and COX1; activity level of citrate synthase; rate of oxygen (O₂) consumption; rate of adenosine triphosphate production; mitochondrial DNA content, level of heteroplasmy, and any combination thereof.

In an additional aspect, administration of the mitochondrially-enriched target cells is by intravenous, intraperitoneal, intraarterial, intrathecal, and intramuscular administration. In a further aspect, between at least 5×10⁵ to 5×10⁹ mitochondrially-enriched target cells are administered to the subject. In certain aspects, a pharmaceutically acceptable carrier is added to the mitochondrially-enriched target cells prior to administration to the subject. In another aspect, the mitochondrially-enriched target cells express CD45.

In certain aspects, the mitochondrially-enriched target cells have an increased content of at least one mitochondrial protein selected from SDHA and COX1; an increased rate of oxygen (O₂) consumption; an increased activity level of citrate synthase; an increased rate of adenosine triphosphate (ATP) production; an increased mitochondrial DNA content; a lower level of heteroplasmy; or any combination thereof as compared to target cells prior to mitochondrial enrichment.

In another embodiment, the present invention provides a pharmaceutical composition for increasing levels of lymphoid cells in a subject comprising mitochondrially-enriched target cells and a pharmaceutically acceptable carrier, wherein the mitochondrially-enriched target cells are enriched with exogenous mitochondria.

In one aspect, the mitochondrially-enriched target cells are produced by the method comprising obtaining target cells from a subject afflicted with a disease or debilitating disorder or a healthy donor; obtaining exogenous mitochondria from a donor; and producing mitochondrially-enriched target cells by contacting the target cells with the exogenous mitochondria under conditions allowing the exogenous mitochondria to enter the target cells, wherein the mitochondrial content of the mitochondrially-enriched target cells is detectably higher than the mitochondrial content of the target cells.

In certain aspects, the target cells are pluripotent stem cells, embryonic stem cells, induced pluripotent stem cells, mesenchymal stem cells, hematopoietic stem cells, hematopoietic progenitor cells, common myeloid progenitor cells, common lymphoid progenitor cells, CD34+ cells or any combination thereof. In a specific aspect, the target cells are CD34+ cells. In an additional aspect, the target cells are obtained from whole blood, blood fractions, peripheral blood, PBMC, serum, plasma, adipose tissue, oral mucosa, blood, umbilical cord blood or bone marrow. In certain aspects, the target cells are allogenic, autologous or syngeneic to a subject. In some aspects, the isolated mitochondria are autologous.

In one aspect, the exogenous mitochondria are isolated or partially purified frozen-thawed human functional mitochondria.

In an additional aspect, the conditions allowing the exogenous mitochondria to enter the target cells comprise incubating the target cells with the exogenous mitochondria at a ratio of about 0.088-176 mU citrate synthase (CS) activity per 10⁶ cells. In another aspect, the conditions allowing the exogenous mitochondria to enter the target cells comprise incubating the target cells with the exogenous mitochondria for a time ranging from about 0.5 to 30 hours at a temperature ranging from about 16 to 37° C. In an additional aspect, the mitochondrial content of the mitochondrially-enriched target cells is determined by assays selected from the group consisting of: content of at least one mitochondrial protein selected from SDHA and COX1; activity level of citrate synthase; rate of oxygen (O₂) consumption; rate of adenosine triphosphate production; mitochondrial DNA content and any combination thereof.

In a further aspect, the mitochondrially-enriched target cells have an increased content of at least one mitochondrial protein selected from SDHA and COX1; an increased rate of oxygen (O₂) consumption; an increased activity level of citrate synthase; an increased rate of adenosine triphosphate (ATP) production; an increased mitochondrial DNA content; a lower level of heteroplasmy; or any combination thereof, as compared to target cells prior to mitochondrial enrichment.

In one aspect, the subject has a disease or disorder. In an additional aspect, the disease or disorder is age related disorders, cancer, muscle diseases and disorders, glycogen-storage diseases and disorders, vascular endothelium disorder or diseases, brain disorder or brain disease, placental disorder or placental disease, thymus disorder or thymus disease, autoimmune diseases, renal disease or renal disorder, primary mitochondrial disease, pancreas disorder or pancreas disease, prostate disorder or prostate disease, kidney disorder or kidney disease, blood disorder or blood disease, heart disease or heart disorder, skin disorder or skin disease, immune and inflammatory diseases and disorders, bone disease or bone disorder, gastro-intestinal disease or gastro-intestinal disorder, eye disease or eye disorder or an infection.

In one aspect, the pharmaceutical composition is administered to the subject. In certain aspects, the pharmaceutical composition is administered to the subject by intravenous, intraperitoneal, intraarterial, intrathecal, and intramuscular. In an additional aspect, between at least 5×10⁵ to 5×10⁹ mitochondrially-enriched target cells are administered.

In one embodiment, the present invention provides a method for diminishing debilitating effects of a lymphocyte deficiency -related disease or diseases in a subject including incubating hematopoietic stem cells (HSCs) with exogenous mitochondria under conditions allowing the exogenous mitochondria to enter the HSCs and administering the HSCs to the subject. In certain aspects, the HSCs are autologous or allogenic stem cells. In a further aspect, the exogenous mitochondria have undergone at least one freeze-thaw cycle. In various aspects, the HSCs are washed prior to administration to the subject.

Prior to bone marrow or hematopoietic stem cell transplantation subjects may go through a conditioning process to eliminate underlying disease and/or to prevent rejection of the new cells.

In an additional embodiment, the present invention provides a method for improving hematopoietic stem cell (HSC) transplantation in a subject including incubating of hematopoietic stem cells (HSCs) with exogenous mitochondria under conditions allowing the exogenous mitochondria to enter the HSCs and administering the HSCs to the subject. In certain aspects, the HSCs are autologous or allogenic stem cells. In an additional aspect, the exogenous mitochondria are isolated from a donor. In a further aspect, the exogenous mitochondria have undergone at least one freeze-thaw cycle. In one aspect, the HSCs are expanded in vitro. In an additional aspect, the HSCs have undergone at least one freeze-thaw cycle. In a further aspect, the HSCs have undergone at least one freeze thaw cycle prior to or following in vitro expansion. In certain aspects, the HSCs have undergone at least one freeze thaw cycle prior to or following incubation with the exogenous mitochondria. In an additional aspect, the conditions allowing the exogenous mitochondria to enter the target cells may include incubating the target cells with the exogenous mitochondria at a ratio of about 0.088-176 mU citrate synthase (CS) activity per 10⁶ cells.

In various aspects, the HSCs are washed prior to administration to the subject.

In a further embodiment, the present invention provides a pharmaceutical composition for enhancing engraftment of cells for gene therapy in a subject comprising mitochondrially-enriched target cells and a pharmaceutically acceptable carrier, wherein the mitochondrially-enriched target cells are enriched with exogenous mitochondria. In one aspect, the target cells have been genetically modified prior to, during or after enrichment with the exogenous mitochondria.

In one embodiment, the present invention provides a method for treating immunodeficiency or immune related diseases in a subject by incubating of hematopoietic stem cells (HSCs) with exogenous mitochondria under conditions allowing the exogenous mitochondria to enter the HSCs; and administering the HSCs to the subject. In certain aspects, the HSCs are autologous or allogenic stem cells. In an additional aspect, the exogenous mitochondria are isolated from a donor. In a further aspect, the exogenous mitochondria have undergone at least one freeze-thaw cycle. In one aspect, the HSCs are expanded in vitro. In an additional aspect, the HSCs have undergone at least one freeze-thaw cycle. In a further aspect, the HSCs have undergone at least one freeze thaw cycle prior to or following in vitro expansion. In certain aspects, the HSCs have undergone at least one freeze thaw cycle prior to or following incubation with the exogenous mitochondria. In an additional aspect, the conditions allowing the exogenous mitochondria to enter the target cells may include incubating the target cells with the exogenous mitochondria at a ratio of about 0.088-176 mU citrate synthase (CS) activity per 10⁶ cells.

In one embodiment the present invention provides a method of treating a disease or disorder by producing mitochondrially-enriched cells by contacting cells with exogenous mitochondria under conditions allowing the exogenous mitochondria to enter the cells; transducing the mitochondrially-enriched cell with a viral vector with a gene of interest; and administering the mitochondrially-enriched transduced cells to a subject. In one embodiment, the present invention provides a method of treating a disease or disorder by transducing cells with a viral vector comprising a gene of interest; producing mitochondrially-enriched cells by contacting the transduced cells with exogenous mitochondria under conditions allowing the exogenous mitochondria to enter the cells; and administering the mitochondrially-enriched transduced cells to a subject. In one aspect, the cells are stem cells. In certain aspects, the cell are hematopoietic stem cells (HSC) or immunodeficient cells. In some aspects, the viral vector is an adeno-associated virus (AAV) vector or a lentivirus vector. In an additional aspect, the administration of the mitochondrially-enriched transduced cell increases the number of B cells compared to non-augmented cells. In certain aspects, the B cells are pre-B or pro-B cells. In a further aspect, the administration of the mitochondrially-enriched transduced cells increases the number of IgM positive cells compared to non-augmented cells. In one embodiment, mitochondrial enrichment increases the number of transduced cells.

### BRIEF DESCRIPTION OF THE DRAWINGS

FIGS. 1A-C show the analysis of the effect of mitochondria enrichment on engraftment in bone marrow two months after transplantation of mitochondria enriched CD34+ cells and non-enriched CD34+ cells into NSGS mice. FIG. 1A. human mitochondria copy number in bone marrow. FIG. 1B. Relative number of human cells in bone marrow. FIG. 1C. Relative human mitochondria copies per cell in bone marrow.

FIGS. 2A-C show the analysis of the effect of mitochondria enrichment on mitochondrial and cellular content in peripheral blood two months after transplantation of mitochondria enriched CD34+ cells and non-enriched CD34+ cells into NSGS mice. FIG. 2A. human mitochondria copy number in peripheral blood. FIG. 2B. Relative number of human cells in peripheral blood. FIG. 2C. Relative human mitochondria copies per cell in peripheral blood.

FIGS. 3A-B show analysis of bone marrow six months post transplantation of mitochondria enriched CD34+ cells into NSGS mice. FIG. 3A: flow cytometry analysis of the percent of human CD45+ cells from the total of human CD45+ cells and mouse CD45+ cells, and the relative percent of CD3+ cells and CD33+ cells from the human CD45+ cells. FIG. 3B: bar graph showing the number of exogenous human mitochondria copies.

FIG. 4 shows flow cytometry analysis of bone marrow six months post transplantation of mitochondria enriched CD34+ cells and non-enriched CD34+ cells into NSGS mice.

FIGS. 5A-B show the frequency of CD45+ cell subsets with peripheral blood. FIG. 5A: flow cytometry analysis of bone marrow and peripheral blood cells at specific time points following mitochondrial augmentation therapy (MAT). FIG. 5B: frequency of CD45+ cell subsets with peripheral blood at specific time points following MAT.

FIG. 6 shows relative levels of exogenous mitochondria that were transferred to recipient cells.

FIG. 7 shows CD3+ (T cells), CD19+ (B cells), CD11b+ (myeloid cells) expression. At the 4.5 m time-point, the myeloid population was further characterized to monocyte (Ly6C^{high}Ly6G⁻) and neutrophil (Ly6C⁺Ly6G⁺) subsets.

FIG. 8 shows that monocytes and neutrophils take up the exogenous mitochondria.

FIG. 9 shows the experimental scheme for testing B-cell development of immunodeficient cells that were augmented and transduced with a BTK gene.

FIG. 10 shows the absolute number of cells at day 13 for augmented and non-augmented cells transduced with NTX101 or NTX109, augmented non-transduced cells, non-augmented non-transduced cells, and wild type (WT) cells.

FIGS. 11A-B show the percentage and absolute number of HSPC population. FIG. 11A. flow cytometry analysis of the percent of HSPC population within the augmented and non-augmented cells transduced with NTX101 or NTX109, augmented non-transduced cells, non-augmented non-transduced cells, and wild type cells. FIG. 11B. absolute number of HSPC population within the NTX101 and NTX109 augmented and non-augmented cells, non-transduced augmented and nonaugmented cells, and wild type cells.

FIG. 12 shows flow cytometry analysis of the percent of pro-B and pre-B cell population within the augmented and non-augmented cells transduced with NTX101 or NTX109, augmented non-transduced cells, non-augmented non-transduced cells, and wild type cells, thirteen days after augmentation.

FIGS. 13A-B show the pro B/pre B cell population ratio and pro B and pre B population absolute cell number, thirteen days after augmentation. FIG. 13A. the pro B/pre B cell population ratio in augmented and non-augmented cells transduced with NTX101 or NTX109, augmented non-transduced cells, nonaugmented non-transduced cells and wild type cells, thirteen days after augmentation. FIG. 13B. the absolute cell number of pro B and pre B cell population in augmented and non-augmented cells transduced with NTX101 and NTX109, augmented non-transduced cells, nonaugmented non-transduced cells and wild type cells, thirteen days after augmentation.

FIG. 14 shows flow cytometry analysis of the percentage and absolute number of B cells within augmented and non-augmented cells transduced with NTX101 or NTX109, augmented non-transduced cells, nonaugmented non-transduced cells and wild type cells, seventeen days after augmentation.

FIG. 15 shows flow cytometry analysis of the IgM positive B cell population within the augmented and non-augmented cells transduced with NTX101 or NTX109, augmented non-transduced cells, nonaugmented non-transduced cells and wild type cells, seventeen days after augmentation.

FIG. 16 shows flow cytometry analysis of GFP expression within the augmented and non-augmented cells transduced with NTX101 or NTX109, augmented non-transduced cells, non-augmented non-transduced cells and wild type cells, thirteen days after augmentation.

FIG. 17 shows flow cytometry analysis of GFP expression within the augmented and non-augmented cells transduced with NTX101 or NTX109, augmented non-transduced cells, non-augmented non-transduced cells, seventeen days after augmentation.

FIG. 18 shows transgene expression for augmented and non-augmented cells transduced with a lentivector (Xid^{pTC9}).

FIG. 19 shows flow cytometry data for gene expression in control cells, non-augmented transduced cells and augmented transduced cells.

### DETAILED DESCRIPTION OF THE INVENTION

The present invention is based on the seminal discovery that cells enriched with exogenous mitochondria are useful for treating diseases and disorders. The present invention provides methods and compositions which cause the bone marrow to increase production of leukocytes cells in a subject by providing mitochondrially-enriched cells. Further the present application provides methods for increasing bone marrow cellularity, engraftment of CD34+ cells and differentiation.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The present invention provides cellular platforms, more specifically stem cell-derived cellular platforms, for targeted and systemic delivery of therapeutically-significant amounts of fully isolated mitochondria and methods for their utilization in a subject. The present invention is based on several surprising findings showing that intravenous injection of bone marrow-derived hematopoietic stem cells enriched with exogenous mitochondria can beneficially affect various tissues of the subject. In other words, improvement in function can be achieved in various organs and tissues following the administration of stem cells enriched with exogenous mitochondria.

The present invention is based in part on the finding that stem cells and bone marrow cells are receptive to being enriched with intact exogenous mitochondria and that human bone marrow cells are particularly receptive to being enriched with mitochondria as disclosed for example in WO 2016/135723. Without being bound to any theory or mechanism, it is postulated that co-incubation of stem cells or bone marrow cells with exogenous mitochondria promotes the transition of intact functional mitochondria into the stem cells or bone marrow cells.

It has also been found that the extent of enrichment of stem cells or bone marrow cells, including but not limited to bone marrow-derived hematopoietic stem cells, with mitochondria and improvement in the cells' mitochondrial functionality are dependent on conditions used for mitochondrial enrichment, including but not limited to the concentration of the isolated exogenous or partially purified mitochondria, as well as the incubation.

The present invention provides in certain aspects, methods and compositions for improving the engraftment of mitochondrially-enriched target cells, proliferation, and the homing of these augmented cells to the bone marrow. Specifically, target cells that have been augmented with exogenous mitochondria have improved engraftment and improved directing of the augmented cells to the bone marrow of a subject as compared to a subject who has received target cells that have not been augmented or enriched with exogenous mitochondria or as compared to the target cells prior to enrichment or augmentation.

The present invention provides, in one aspect, methods and compositions for increasing levels of CD45+ cells, increasing the level of CD3+ cells relative to CD33+ cells, increasing the proportion of lymphoid cells to myeloid cells in a subject, by enriching target cells obtained or derived from a subject afflicted with a disease or disorder or from a healthy subject with exogenous mitochondria, and transplanting the “mitochondrially-enriched” target cells into the subject.

The present invention provides, a method for increasing the levels of lymphoid cells in a subject, the method includes obtaining target cells from a subject afflicted with or having a disease or disorder or from a healthy subject; obtaining exogenous mitochondria from a donor; producing mitochondrially-enriched target cells by contacting the target cells with the exogenous mitochondria under conditions allowing the exogenous mitochondria to enter the target cells; and administering the mitochondrially-enriched target cell to the subject, wherein the mitochondrial content of the enriched target cells is detectably higher than the mitochondrial content of the target cells. In one aspect, the conditions for allowing the exogenous mitochondria to enter the target cells comprises incubating the target cells with the exogenous mitochondria at a ratio of about 0.088-176 mU citrate synthase (CS) activity per 10⁶ per target cells.

The present invention additionally provides for pharmaceutical compositions for increasing levels of lymphoid cells in a subject comprising mitochondrially-enriched target cells and a pharmaceutically acceptable carrier, wherein the mitochondrially-enriched target cells are enriched with exogenous mitochondria. The mitochondrially-enriched target cells are produce by obtaining target cells from a subject afflicted with or having a disease or disorder or from a healthy subject; obtaining exogenous mitochondria from a donor; and producing mitochondrially-enriched target cells by contacting the target cells with the exogenous mitochondria under conditions allowing the exogenous mitochondria to enter the target cells; wherein the mitochondrial content of the enriched target cells is detectably higher than the mitochondrial content of the target cells. In one aspect, the conditions for allowing the exogenous mitochondria to enter the target cells comprises incubating the target cells with the exogenous mitochondria at a ratio of about 0.088-176 mU citrate synthase (CS) activity per 10⁶ per target cells.

Lymphoid cells or lymphocytes are white blood cells that provide an immune response to antigens. Lymphoid cells include T cells, B cells and natural killer (NK) cells. T cells and B cells are the major cellular components of the adaptive immune response. T cells are involved in cell-mediated immunity, whereas B cells are primarily responsible for humoral immunity. NK cells are a part of the innate immune system and play a major role in defending the host from tumors and virally infected cells.

As used herein the term “increased levels of leukocyte cells” refers to a subject having been administered mitochondrially-enriched target cells and have an increase in the number of leukocyte cells compared to a subject who has not been administered mitochondrially-enriched target cells, compared to a subject who has been administered target cells that were not mitochondrially enriched, or compared to the level of leukocyte cells in the subject prior to administration of mitochondrially-enriched target cells. According to some embodiments, levels of CD45+ cells are increased by at least 1.1 fold, 1.3 fold, 1.5 fold, 2 fold, at least 2.5 fold, at least 3 fold, at least 3.5 fold, at least 4 fold, at least 4.5 fold or at least 5 fold.

As used herein the term “increased levels of lymphoid cells” refers to a subject having been administered mitochondrially-enriched target cells and have an increase in the number of lymphoid cells compared to a subject who has not been administered mitochondrially-enriched target cells, compared to a subject who has been administered target cells that were not mitochondrially enriched, or compared to the level of lymphoid cells in the subject prior to administration of mitochondrially-enriched target cells. In certain aspects, levels of CD3+ cells are increased by at least 1.1 fold, 1.3 fold, 1.5 fold, 2 fold, at least 2.5 fold, at least 3 fold, at least 3.5 fold, at least 4 fold, at least 4.5 fold or at least 5 fold.

As used herein the term “increased proportion of lymphoid cells to myeloid cells” refers to a subject having been administered mitochondrially-enriched target cells and have an increase in the proportion of lymphoid cells to myeloid cells compared to a subject who has not been administered mitochondrially-enriched target cells, compared to a subject who has been administered target cells that were not mitochondrially enriched, or compared to the proportion of lymphoid cells to myeloid cells in the subject prior to administration of mitochondrially-enriched target cells. In certain aspects, the increase is at least 1.1 fold, 1.3 fold, 1.5 fold, 2 fold, at least 2.5 fold, at least 3 fold, at least 3.5 fold, at least 4 fold, at least 4.5 fold, or at least 5 fold.

As used herein the term “target cell” is stem cell, progenitor cell or bone marrow derived stem cell. Specifically, a target cell includes pluripotent stem cells, embryonic stem cells, induced pluripotent stem cells, mesenchymal stem cells, hematopoietic stem cells, hematopoietic progenitor cells, common myeloid progenitor cells, common lymphoid progenitor cells, CD34+ cells and any combination thereof. In the methods of the invention, the target cells are not actively altered or modified (e.g., reduction of mtDNA or mitochondrial function) prior to enrichment with exogenous mitochondria. More specifically, mitochondrial function and/or mitochondria DNA in the target cells are not actively altered or modified prior to contacting with exogenous mitochondria.

As used herein, the term “stem cells” generally refers to any mammalian stem cells. Stem cells are undifferentiated cells that can differentiate into other types of cells and can divide to produce more of the same type of stem cells. Stem cells can be either totipotent or pluripotent.

As used herein, the term “human stem cells” generally refers to all stem cells naturally found in humans, and to all stem cells produced or derived ex vivo and are compatible with humans. In some embodiments, the human stem cells are autologous. In some embodiments, the human stem cells are allogeneic. A “progenitor cell”, like a stem cell, has a tendency to differentiate into a specific type of cell, but is already more specific than a stem cell and is pushed to differentiate into its “target” cell. The most important difference between stem cells and progenitor cells is that stem cells can replicate indefinitely, whereas progenitor cells can divide only a limited number of times. The term “human stem cells” as used herein further includes “progenitor cells” and “non-fully differentiated stem cells”.

HSPCs are the progenitors of all immune cells, innate and adaptive immune systems, and cells of myeloid and lymphoid lineages. As stem cells, they are long lived; as hematopoietic cells, they circulate systemically and affect every organ system in the body, key to addressing multi-systemic diseases. Mitochondrial metabolism is crucial for hematopoietic stem and progenitor cell persistence and function, as well as for function and inflammatory balance of immune cells. Immune dysfunction is a major cause of morbidity and mortality in mitochondrial disease patients and is known to result in neurodegenerative sequalae. The immune system is affected by energetic deficits and mitochondrial disease patients frequently have presentations similar to those of patients with primary immunodeficiencies including unusual infections not generally seen in immunocompetent populations. Importantly, even in patients without transfusion dependence or cytopenias, higher risk of metabolic decompensations during infections exists, this may be due to cell intrinsic defects in immune cell mitochondrial function.

Preclinically, it was recently demonstrated that rescue of immune cell mitochondrial function can exert multisystemic effects, including improvement of anemia, muscle wasting, physical activity, cardiovascular function, and reduction of tissue senescence. Suggested mechanisms by which hematopoietic cells exert non-hematopoietic effects include reduction of inflammation associated with mitochondrial dysfunction in immune cells or enhanced secretion of factors capable of suppression of apoptosis in distal tissues.

In certain embodiments, the stem cells are pluripotent stem cells (PSC). In other embodiments, the PSCs are non-embryonic stem cells. According to some embodiments embryonic stem cells are explicitly excluded from the scope of the invention. In some embodiments, the stem cells are induced PSCs (iPSCs). In certain embodiments, the stem cells are embryonic stem cells. In certain embodiments, the stem cells are derived from bone-marrow cells. In particular embodiments, the stem cells are CD34+ cells. In particular embodiments the stem cells are mesenchymal stem cells. In other embodiments, the stem cells are derived from adipose tissue. In yet other embodiments, the stem cells are derived from blood. In further embodiments, the stem cells are derived from umbilical cord blood. In further embodiments the stem cells are derived from oral mucosa. In specific embodiments, the stem cells obtained from a patient afflicted with a disease of disorder or from a healthy subject are bone marrow cells or bone marrow-derived stem cells. Each possibility represents a separate embodiment of the present invention.

As used herein the term “pluripotent stem cells (PSCs)” refers to cells that can propagate indefinitely, as well as give rise to a plurality of cell types in the body. Totipotent stem cells are cells that can give rise to every other cell type in the body. Embryonic stem cells (ESCs) are totipotent stem cells and induced pluripotent stem cells (iPSCs) are pluripotent stem cells.

As used herein the term “induced pluripotent stem cells (iPSCs)” refers to a type of pluripotent stem cell that can be generated from human adult somatic cells. Some non-limiting examples of somatic cells from which iPSC can be generated herein include fibroblast cells, endothelial cells, capillary blood cells, keratinocytes, myeloid cells epithelial cells.

As used herein the term “embryonic stem cells (ESC)” refers to a type of totipotent stem cell derived from the inner cell mass of a blastocyst.

As used herein the term “bone marrow cells” generally refers to all human cells naturally found in the bone marrow of humans, and to all cell populations naturally found in the bone marrow of humans. The term “bone marrow stem cells” and “bone marrow-derived stem cells” refer to the stem cell population derived from the bone marrow.

In some embodiments, the target cells are pluripotent stem cells, embryonic stem cells, induced pluripotent stem cells, mesenchymal stem cells, hematopoietic stem cells, hematopoietic progenitor cells, common myeloid progenitor cells, common lymphoid progenitor cells, CD34+ cells and any combination thereof.

In some embodiments, the autologous or allogeneic human stem cells are pluripotent stem cells (PSCs) or induced pluripotent stem cells (iPSCs). In further embodiments, the autologous or allogeneic human stem cells are mesenchymal stem cells.

According to several embodiments, the human stem cells are derived from adipose tissue, oral mucosa, blood, umbilical cord blood or bone marrow. Each possibility represents a separate embodiment of the present invention. In specific embodiments, the human stem cells are derived from bone marrow.

In certain embodiments, the bone-marrow derived stem cells include myelopoietic cells.

The term “myelopoietic cells” as used herein refers to cells involved in myelopoiesis, e.g. in the production of bone-marrow and of all cells that arise from it, namely, all blood cells.

In certain embodiments, the bone-marrow derived stem cells include erythropoietic cells. The term “erythropoietic cells” as used herein refers to cells involved in erythropoiesis, e.g. in the production of red blood cells (erythrocytes).

In certain embodiments, the bone-marrow derived stem cells include multi-potential hematopoietic stem cells (HSCs). The term “multi-potential hematopoietic stem cells” or “hemocytoblasts” as used herein refers to the stem cells that give rise to all the other blood cells through the process of hematopoiesis.

In certain embodiments, the bone-marrow derived stem cells comprise common myeloid progenitor cells, common lymphoid progenitor cells, or any combination thereof. In certain embodiments, the bone-marrow derived stem cells comprise mesenchymal stem cells. The term “common myeloid progenitor” as used herein refers to the cells that generate myeloid cells. The term “common lymphoid progenitor” as used herein refers to the cells that generate lymphocytes.

In certain embodiments, the bone-marrow derived stem cells further comprise megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. Each possibility represents a separate embodiment of the invention.

In certain embodiments, the bone-marrow derived stem cells include mesenchymal stem cells. The term “mesenchymal stem cells” as used herein refers to multipotent stromal cells that can differentiate into a variety of cell types, including osteoblasts, chondrocytes, myocytes and adipocytes.

In certain embodiments, the bone-marrow derived stem cells include myelopoietic cells. In certain embodiments, the bone-marrow derived stem cells consist of erythropoietic cells. In certain embodiments, the bone-marrow derived stem cells include multi-potential hematopoietic stem cells (HSCs). In certain embodiments, the bone-marrow derived stem cells include common myeloid progenitor cells, common lymphoid progenitor cells, or any combination thereof. In certain embodiments, the bone-marrow derived stem cells include megakaryocytes, erythrocytes, mast cells, myoblasts, basophils, neutrophils, eosinophils, monocytes, macrophages, natural killer (NK) cells, small lymphocytes, T lymphocytes, B lymphocytes, plasma cells, reticular cells, or any combination thereof. In certain embodiments, the bone-marrow derived stem cells consist of mesenchymal stem cells. In certain embodiments, the stem cells include a plurality of human bone marrow stem cells obtained from peripheral blood.

Hematopoietic progenitor cell antigen CD34, also known as CD34 antigen, is a protein that in humans is encoded by the CD34 gene. CD34 is a cluster of differentiation in a cell surface glycoprotein and functions as a cell-cell adhesion factor. In certain embodiments, the bone-marrow stem cells express the bone-marrow progenitor cell antigen CD34 (are CD34+). In certain embodiments, the bone marrow stem cells present the bone-marrow progenitor cell antigen CD34 on their external membrane. In certain embodiments the CD34+ cells are from umbilical cord blood.

As used herein the term “CD34+ cells” refers to hematopoietic stem cells characterized as being CD34 positive, regardless of their origin. In certain embodiments, the CD34+ cells are obtained from the bone marrow, from bone marrow cells mobilized to the blood, or obtained from umbilical cord blood.

As used herein the phrase “stem cells obtained from a subject afflicted with a disorder or from a donor not afflicted with a disorder” refers to cells that were stem cells in the subject/donor at the time of their isolation from the subject.

As used herein the phrase “stem cells derived from a subject afflicted with a disorder” or “from a donor not afflicted with a disorder” refers to cells that were not stem cells in the subject/donor, and have been manipulated to become stem cells. The term “manipulated” as used herein refers to the use of any one of the methods known in the field (Yu J. et al., Science, 2007, Vol. 318(5858), pages 1917-1920) for reprograming somatic cells to an undifferentiated state and becoming induced pluripotent stem cells (iPSCs), and, optionally, further reprograming the iPSCs to become cells of a desired lineage or population (Chen M. et al., IOVS, 2010, Vol. 51(11), pages 5970-5978), such as bone marrow cells (Xu Y. et al., PLoS ONE, 2012, Vol. 7(4), page e34321).

In some embodiments, the stem cells are cultured and expanded in vitro. In certain embodiments, the stem cells are undergo at least one freeze thaw cycle prior to or following mitochondrial enrichment.

In certain embodiments, the stem cells are directly derived from the subject afflicted with a disease or disorder. In certain embodiments, the stem cells are directly derived from a donor.

The term “directly derived” as used herein refers to stem cells which were derived directly from other cells. In certain embodiments, the hematopoietic stem cells (HSC) were derived from bone-marrow cells. In certain embodiments, the hematopoietic stem cells (HSC) were derived from peripheral blood.

In certain embodiments, the stem cells are indirectly derived from the subject afflicted with a disease or disorder. In certain embodiments, the stem cells are indirectly derived from a donor. The term “indirectly derived” as used herein refers to stem cells which were derived from non-stem cells. In certain embodiments, the stem cells were derived from somatic cells which were manipulated to become induced pluripotent stem cells (iPSCs).

In some embodiments, the target cells are obtained from whole blood, blood fractions, peripheral blood, PBMC, serum, plasma, adipose tissue, oral mucosa, blood, umbilical cord blood or bone marrow. In certain embodiments, the stem cells are directly obtained from the bone marrow of the subject afflicted with a disease or disorder. In certain embodiments, the stem cells are directly obtained from the bone-marrow of a donor. The term “directly obtained” as used herein refers to stem cells which were obtained from the bone-marrow itself, e.g. by means such as surgery or suction through a needle by a syringe.

In certain embodiments, the stem cells are indirectly obtained from the bone marrow of the patient afflicted with a disease or disorder. In certain embodiments, the stem cells are indirectly obtained from the bone marrow of a donor. The term “indirectly obtained” as used herein refers to bone marrow cells which were obtained from a location other than the bone marrow itself.

In certain embodiments, the stem cells are obtained from the peripheral blood of the subject afflicted with a disease or disorder. In certain embodiments, the stem cells are obtained from the peripheral blood of a healthy. The term “peripheral blood” as used herein refers to blood circulating in the blood system.

As used herein, the term “autologous cells” or “cells that are autologous”, refers to being the patient's own cells. The term “autologous mitochondria”, refers to mitochondria obtained from the patient's own cells or from maternally related cells. The terms “allogeneic cells” or “allogeneic mitochondria”, refer to being from a different donor individual.

The term “syngeneic” as used herein and in the claims refers to genetic identity or genetic near-identity sufficient to allow grafting among individuals without rejection. The term syngeneic in the context of mitochondria is used herein interchangeably with the term autologous mitochondria meaning of the same maternal bloodline.

The terms “disease” and “disorder” are meant to refer to any affliction that are not considered normal or that are different from a physiological state. Disease and disorders can affect virtually any organ, tissue, or function is the body. Non limiting examples of diseases and condition include cancer, muscle diseases and disorders, glycogen-storage diseases and disorders, vascular endothelium disorder or diseases, brain disorder or brain disease, placental disorder or placental disease, thymus disorder or thymus disease, autoimmune diseases, renal disease or disorder, pancreas disorder or pancreas disease, prostate disorder or prostate disease, kidney disorder or kidney disease, blood disorder or blood disease, heart disease or heart disorder, skin disorder or skin disease, immune and inflammatory diseases and disorders, bone disease or bone disorder, gastro-intestinal disease or gastro-intestinal disorder, and eye disease or eye disorder. In certain aspects, the disease or disorder is an age related disease or disorder.

An age-related disease is a disease that is most often seen with increasing frequency with increasing cell senescence. Essentially, age-related diseases are complications arising from senescence. Age-related diseases are to be distinguished from the aging process itself because all adult animals age, but not all adult animals experience age-related diseases.

A decline in mitochondrial quality and activity has been associated with normal aging and correlated with the development of a wide range of age-related diseases. Mitochondria contribute to specific aspects of the aging process, including cellular senescence, chronic inflammation and the age-dependent decline in stem cell activity. A wealth of supportive evidence demonstrates that mitochondrial dysfunction occurs with age due to accumulation of mitochondrial DNA mutations. Various mitochondrial DNA point mutations have been shown to significantly increase with age in the human brain, heart, skeletal muscles and liver tissues. Increased frequency of mitochondrial DNA deletions/insertions have also been reported with increasing age in both animal models and humans. It has been postulated that the replication cycle and the accumulation of mitochondrial DNA mutations might be a conserved mechanism underlying stem cell aging such that mitochondria influence or regulate a number of key aspects of aging (Sun et al., Cell, 2016, 61: 654-66; Srivastava, Genes, 2017, 8:398; Ren et al., Genes, 2017, 8:397).

As used herein, the term “mitochondrial disease” and the term “primary mitochondrial disease” may be used interchangeably. The term “primary mitochondrial disease” as used herein refers to a mitochondrial disease which is diagnosed by a known or indisputably pathogenic mutation in the mitochondrial DNA, or by mutations in genes of the nuclear DNA, whose gene products are imported into the mitochondria. According to some embodiments, the primary mitochondrial disease is a congenital disease. According to some embodiments, the primary mitochondrial disease is not a secondary mitochondrial dysfunction. The terms “secondary mitochondrial dysfunction” and “acquired mitochondrial dysfunction” are used interchangeably throughout the application.

As used herein the term “a subject afflicted with a disease or disorder” or “a subject afflicted having a disease or disorder” refers to a human subject experiencing debilitating effects caused by certain conditions. The disorder may refer to cancer, age related disorders, renal disease, pancreatic diseases, liver diseases, muscle disorders, brain disease or primary mitochondrial diseases, secondary mitochondrial dysfunction, as well as other disease or disorders.

As used herein, the term “ex-vivo method” refers to a method where the steps are performed exclusively outside the human body. In particular, an ex vivo method includes manipulation of cells outside the body that are subsequently reintroduced or transplanted into the subject to be treated.

As used herein the term “donor” refers to a donor providing the exogenous mitochondria. In some embodiments, the donor is not suffering from a disease or disorder or is not suffering from the same disease of disorder which the subject is afflicted.

The term “exogenous” or “isolated exogenous” with regard to mitochondria refers to mitochondria that are introduced to a target cell (for example, stem cells), from a source which is external to the cell. For example, in some embodiments, exogenous mitochondria are commonly derived or isolated from a donor cell which is different than the target cell. For example, exogenous mitochondria may be produced or made in a donor cell, purified, isolated or obtained from the donor cell and thereafter introduced into the target cell. Exogenous mitochondria can be allogenic as obtained from a donor or autologous as obtained from a subject. Isolated mitochondria may include functional mitochondria. In certain embodiments, the exogenous mitochondria are whole mitochondria.

As used herein, the terms “isolated” and “partially purified” in the context of mitochondria includes exogenous mitochondria that were purified, at least partially, from other cellular components. The total amount of mitochondrial proteins in an exogenous isolated or partially purified mitochondria is between about 10%-90% of the total amount of cellular proteins within the sample.

As used herein the term “functional mitochondria” refers to mitochondria displaying parameters indicative of normal mitochondrial DNA (mtDNA) and normal, non-pathological levels of activity. The activity of mitochondria can be measured by a variety of methods well known in the art, such as membrane potential, O₂ consumption, ATP production, and citrate synthase (CS) activity level.

In certain embodiments, the exogenous mitochondria constitute at least 1% of the total mitochondria content in the mitochondrially-enriched cell. In certain embodiments, the exogenous mitochondria constitute at least 10% of the total mitochondria content in the mitochondrially-enriched target cell. In some embodiments, the exogenous mitochondria constitute at least about 3%, 5%, 10%, 15%, 20%, 25%,30%, 40% or 50% of the total mitochondria content in the mitochondrially-enriched target cell. In certain embodiments, the total amount of mitochondrial proteins in the isolated mitochondria, is between 10-90%, 20-80%, 20-70%, 40-70%, 20-40%, or 20-30% of the total amount of cellular proteins. Each possibility represents a separate embodiment of the present invention. In certain embodiments, the total amount of mitochondrial proteins in the isolated mitochondria, is between 20%-80% of the total amount of cellular proteins within the sample. In certain embodiments, the total amount of mitochondrial proteins in the isolated mitochondria, is between 20%-80% of the combined weight of the mitochondria and other sub-cellular fractions. In other embodiments, the total amount of mitochondrial proteins in the isolated mitochondria, is above 80% of the combined weight of the mitochondria and other sub-cellular fractions.

In certain embodiments, the exogenous mitochondria are obtained from a human cell or a human tissue. In some embodiments, the human cell or human tissue is selected from the group consisting of placenta, placental cells grown in culture, and blood cells. In some embodiments, the human cell is a human stem cell. In some embodiments, the human cell is a human somatic cell. In some embodiments, the cells are cells in culture. Some non-limiting examples of somatic cells from which iPSC can be generated herein include fibroblast cells, endothelial cells, capillary blood cells, keratinocytes, myeloid cells, and epithelial cells.

The term “autologous” with regards to mitochondria refers to mitochondria that are introduced to a target cell (for example, stem cells), from a source which is the same as the cell. For example, in some embodiments, autologous mitochondria are derived or isolated from a subject that is the source of the target cell. For example, autologous mitochondria may be purified/isolated/obtained from the subject's cell and thereafter introduced into the target cell of the subject.

The term “endogenous” with regard to mitochondria refers to mitochondria that is being made/expressed/produced by a cell and is not introduced from an external source into the cell. In some embodiments, endogenous mitochondria contain proteins and/or other molecules which are encoded by the genome of the cell. In some embodiments, the term “endogenous mitochondria” is equivalent to the term “host mitochondria”.

According to the principles of the present invention, exogenous human mitochondria are introduced into target cells which may be human stem cells, thus enriching these cells with exogenous mitochondria. It should be understood that such enrichment changes the mitochondrial content of the target cells: while naive human stem cells substantially have one population of host/autologous mitochondria, target cells enriched with exogenous mitochondria substantially have two populations of mitochondria, a first population of host/ endogenous mitochondria and another population of the introduced mitochondria (i.e., the exogenous mitochondria). Thus, the term “enriched” relates to the state of the cells after receiving/incorporation exogenous mitochondria. Determining the number and/or ratio between the two populations of mitochondria is straightforward, as the two populations may differ in several aspects e.g. in their mitochondrial DNA. Therefore, the phrase “human stem cells enriched with exogenous human mitochondria” is equivalent to the phrase “human stem cells comprising endogenous mitochondria and exogenous isolated mitochondria”. For example, human stem cells which comprise at least 1% exogenous isolated mitochondria of the total mitochondria content, are considered comprising host endogenous mitochondria and exogenous isolated mitochondria in a ratio of 99:1. For example,“3% of the total mitochondria” means that after enrichment the original (endogenous) mitochondrial content is 97% of the total mitochondria and the introduced (exogenous) mitochondria is 3% of the total mitochondria—this is equivalent to (3/97=) 3.1% enrichment. Another example −“33% of the total mitochondria” means that after enrichment, the original (endogenous) mitochondrial content is 67% of the total mitochondria and the introduced (exogenous) mitochondria is 33% of the total mitochondria—this is equivalent to (33/67=) 49.2% enrichment.

In some embodiments, the identification/discrimination of endogenous mitochondria from exogenous mitochondria, after the latter have been introduced into the target cell, can be performed by various means, including, for example, but not limited to: identifying differences in mtDNA sequences, for example different haplotypes, between the endogenous mitochondria and exogenous mitochondria, identifying specific mitochondrial proteins originating from of the source tissue of the exogenous mitochondria, such as, for example, cytochrome p450 cholesterol side chain cleavage (P450SCC) from placenta, UCP1 from brown adipose tissue, and the like, or any combination thereof.

Heteroplasmy is the presence of more than one type of mitochondrial DNA within a cell or individual. The heteroplasmy level is the proportion of mutant mtDNA molecules vs. wild type/functional mtDNA molecules and is an important factor in considering the severity of mitochondrial diseases. While lower levels of heteroplasmy (sufficient amount of mitochondria are functional) are associated with a healthy phenotype, higher levels of heteroplasmy (insufficient amount of mitochondria are functional) are associated with pathologies. In certain embodiments, the heteroplasmy level of the enriched stem cells is at least 1%, 3%, 5%, 15%, 20%, 25%, or 30% lower than the heteroplasmy level of the stem cells obtained or derived from the subject or donor.

As used herein the term “mitochondrially-enriched target cells” refers to a target cell that has had exogenous mitochondria inserted. In certain embodiments, the mitochondrially enriched target cells differentiate to CD45, CD3, CD33, CD14, CD19, CD11, CD15, CD16 and the like expressing cells. In certain embodiments, the mitochondrially enriched target cells express CD45, CD3, CD33, CD14, or CD19. CD45 is a receptor linked protein tyrosine phosphatase present in all cells of the hematopoietic lineage except erythrocytes and plasma cells. CD3 is a marker of immune response efficiency. Specifically, CD3 is expressed in pro-thymocytes. Expression of CD45 and CD3 on cells can be determined by any means known in the art including flow cytometry. According to some embodiments, CD45, CD3, CD33, CD14 and/or CD19 expression occurs after the mitochondrially-enriched cells are administered to a subject.

As used herein the term “contacting” refers to bringing the mitochondria and cells into sufficient proximity to promote entry of the mitochondria into the cells. The term introducing or inserting mitochondria into the target cells is used interchangeably with the term contacting.

The phrase “conditions allowing the isolated mitochondria to enter the target cells” as used herein generally refers to parameters such as time, temperature, culture medium and proximity between the mitochondria and the stem cells. For example, human cells and human cell lines are routinely incubated in liquid medium, and kept in sterile environments, such as in tissue culture incubators, at 37° C. and 5% CO₂ atmosphere. According to alternative embodiments disclosed and exemplified herein the cells may be incubated at room temperature in saline supplemented with human serum albumin.

In certain embodiments, the human stem cells are incubated with the isolated mitochondria for a time ranging from about 0.5 to 30 hours, at a temperature ranging from about 16 to about 37° C. In certain embodiments, the human stem cells are incubated with the isolated mitochondria for a time ranging from about 1 to 30 or from about 5 to 25 hours. In specific embodiments, incubation is for about 20 to 30 hours. In some embodiments, incubation is for at least 1, 3, 5, 8, 10, 13, 15, 18, 20, 21, 22, 23 or 24 hours. In other embodiments, incubation is up to at least 5, 10, 15, 20 or 30 hours. In specific embodiments, incubation is for 24 hours. In certain embodiments, incubation is until the mitochondrial content in the target cells is increased in average by about 1% to 45% compared to their initial mitochondrial content.

In some embodiments, incubation is at room temperature (16° C. to 30° C.). In other embodiments, incubation is at 37° C. In some embodiments, incubation is in a 5% CO₂ atmosphere. In other embodiments, incubation does not include added CO₂ above the level found in air.

In yet further embodiments, the incubation is performed in culture medium supplemented with human serum albumin (HSA). In additional embodiments, the incubation is performed in saline supplemented with HSA. According to certain exemplary embodiments, the conditions allowing the isolated exogenous mitochondria to enter the human stem cells thereby enriching said human stem cells with said human exogenous mitochondria include incubation at room temperature in saline supplemented with 4.5% human serum albumin.

In certain embodiments, the isolated mitochondria are incubated with the target cells at about 1, 2, 3, 4, 5, 6, 7, 8, 9, 10, 15, 20, 25, 30, 35, 40, 45, 50, 55 or 60 minutes after the mitochondria are obtained. In an additional embodiment, the isolated mitochondria are incubated with the target cells for about 0.5, 1, 1.5, 2, 2.5, 3, 3.5, 4, 4.5, 5, 6, 7, 8, 9, 10, 11, 12, 13, 14, 15, 16, 17, 18, 19, 20, 21, 22, 23 or 24 hours after the isolated mitochondria are obtained. In one aspect the mitochondria are obtained from a donor. In another aspect the exogenous mitochondria are autologous or allogenic to the target cell.

In certain embodiments, the incubation is performed at 37° C. In certain embodiments, the incubation is performed for at least 6 hours. In certain embodiments, the incubation is performed for at least 12 hours. In certain embodiments, the incubation is performed for 12 to 24 hours. In certain embodiments, the incubation is performed at a ratio of about 1×10⁵ to 1×10⁷ target cells per amount of exogenous mitochondria having or exhibiting 0.88milliunits of citrate synthase (CS). In certain embodiments, the incubation is performed at a ratio of 1×10⁶ naive stem cells per amount of exogenous mitochondria having or exhibiting 0.88milliunits of CS. In certain embodiments, the conditions are sufficient to increase the mitochondrial content of the naive stem cells by at least about 1%, 3%, 5% or 10% as determined by CS activity. Each possibility represents a separate embodiment of the present invention.

As used herein, the term “enriching” refers to any action designed to increase the mitochondrial content, e.g. the number of intact mitochondria, or the functionality of mitochondria of a mammalian cell. In a particular embodiment, stem cells enriched with exogenous mitochondria will show enhanced function compared to the same stem cells prior to enrichment.

Citrate synthase (CS) is localized in the mitochondrial matrix, but is encoded by nuclear DNA. Citrate synthase is involved in the first step of the Krebs cycle, and is commonly used as a quantitative enzyme marker for the presence of intact mitochondria (Larsen S. et al., J. Physiol., 2012, Vol. 590(14), pages 3349-3360; Cook G. A. et al., Biochim. Biophys. Acta., 1983, Vol. 763(4), pages 356-367).

Mitochondrial dose can be expressed in terms of units of CS activity or mtDNA copy number of other quantifiable measurements of the amount of exogenous mitochondria as explained herein. A “unit of CS activity” is defined as the amount that enables conversion of one micromole substrate in 1 minute in 1 mL reaction volume.

In some embodiments, the enrichment of the stem cells with exogenous mitochondria includes introducing into the target cells a dose of mitochondria of at least 0.044 to 176 milliunits (mU) of citrate synthase (CS) activity per million cells; at least 0.088 to 176 mU of CS activity per million cells; at least 0.2 to 150 mU of CS activity per million cells; at least 0.4 to 100 mU of CS activity per million cells; at least 0.6 to 80 mU of CS activity per million cells; at least 0.7 to 50 mU of CS activity per million cells; at least 0.8 to 20 mU of CS activity per million cells; at least 0.88 to 17.6 mU of CS activity per million cells; or at least 0.44 to 17.6 milliunits of CS activity per million cells.

As used herein the term “mitochondrial content” refers to the amount of mitochondria within a cell, or to the average amount of mitochondria within a plurality of cells. The term “increased mitochondrial content” as used herein refers to a mitochondrial content which is detectably higher than the mitochondrial content of the target cells prior to mitochondria enrichment.

In certain embodiments, the mitochondrial content of the human stem cells enriched with exogenous mitochondria is detectably higher than the mitochondrial content of the target cells. According to various embodiments, the mitochondrial content of the mitochondrially-enriched target cells is at least 3%, at least 5%, at least 10%, at least 25%, at least 50%, at least 100%, at least 200% or more, higher than the mitochondrial content of the target cells.

In certain embodiments, the target cells are used fresh. In some embodiments, the target cells are frozen and thawed prior to or following enrichment with mitochondria.

In certain embodiments, the mitochondrial content of the target cells or mitochondrially-enriched target cells is determined by determining the content of citrate synthase. In certain embodiments, the mitochondrial content of the stem cells or enriched stem cells is determined by determining the activity level of citrate synthase. In certain embodiments, the mitochondrial content of the stem cells or enriched stem cells correlates with the content of citrate synthase. In certain embodiments, the mitochondrial content of the stem cells or enriched stem cells correlates with the activity level of citrate synthase. CS activity can be measured by commercially available kits e.g., using the CS activity kit CS0720 (Sigma).

Mitochondrial DNA content may be measured by performing quantitative PCR of a mitochondrial gene prior and post mitochondrial enrichment, normalized to a nuclear gene.

In specific situations the same cells, prior to mitochondria enrichment, serve as controls to measure CS and ATP activity and determine enrichment level.

In certain embodiments, the term “detectably higher” as used herein refers to a statistically-significant increase between the normal and increased values. In certain embodiments, the term “detectably higher” as used herein refers to a non-pathological increase, i.e. to a level in which no pathological symptom associated with the substantially higher value becomes apparent. In certain embodiments, the term “increased” as used herein refers to a value which is about 1.05 fold, 1.1 fold, 1.25 fold, 1.5 fold, 2 fold, 3 fold, 4 fold, 5 fold, 6 fold, 7 fold or higher than the corresponding value found in corresponding cells or corresponding mitochondria of a healthy subject or of a plurality of healthy subjects or in the target cells prior to mitochondrial enrichment. Each possibility represents a separate embodiment of the invention.

The term “increased mitochondrial DNA content” as used herein refers to the content of mitochondrial DNA which is detectably higher than the mitochondrial DNA content in target cells prior to mitochondria enrichment. Mitochondrial content may be determined by measuring SDHA or COX1 content. “Normal mitochondrial DNA” in the context of the specification and claims refers to mitochondrial DNA not carrying/having a mutation or deletion that is known to be associated with a mitochondrial disease. The term “normal rate of oxygen (O₂) consumption” as used herein refers to the average 02 consumption of cells from healthy individuals. The term “normal activity level of citrate synthase” as used herein refers to the average activity level of citrate synthase in cells from healthy individuals. The term “normal rate of adenosine triphosphate (ATP) production” as used herein refers to the average ATP production rate in cells from healthy individuals.

The extent of enrichment of the stem cells with exogenous mitochondria may be determined by functional and/or enzymatic assays, including but not limited to rate of oxygen (O₂) consumption, content or activity level of citrate synthase, rate of adenosine triphosphate (ATP) production. In the alternative the enrichment of the stem cells with exogenous mitochondria may be confirmed by the detection of mitochondrial DNA of the donor. According to some embodiments, the extent of enrichment of the stem cells with exogenous mitochondria may be determined by the level of change in heteroplasmy and/or by the copy number of mtDNA per cell. Each possibility represents a separate embodiment of the present invention.

TMRM (tetramethylrhodamine methyl ester) or the related TMRE (tetramethylrhodamine ethyl ester) are cell-permeant fluorogenic dyes commonly used to assess mitochondrial function in living cells, by identifying changes in mitochondrial membrane potential. According to some embodiments, the level of enrichment can be determined by staining with TMRE or TMRM.

According to some embodiments, the intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Each possibility represents a separate embodiment of the present invention. Mitochondria that were observed under a microscope and show TMRM or TMRE staining have an intact mitochondrial outer membrane. As used herein, the term “a mitochondrial membrane” refers to a mitochondrial membrane selected from the group consisting of the mitochondrial inner membrane, the mitochondrial outer membrane, and both.

In certain embodiments, the level of mitochondrial enrichment in the mitochondrially-enriched human stem cells is determined by sequencing at least a statistically-representative portion of total mitochondrial DNA in the cells and determining the relative levels of host/endogenous mitochondrial DNA and exogenous mitochondrial DNA. In certain embodiments, the level of mitochondrial enrichment in the mitochondrially-enriched human stem cells is determined by single nucleotide polymorphism (SNP) analysis. In certain embodiments, the largest mitochondrial population and/or the largest mitochondrial DNA population is the host/endogenous mitochondrial population and/or the host/endogenous mitochondrial DNA population; and/or the second-largest mitochondrial population and/or the second-largest mitochondrial DNA population is the exogenous mitochondrial population and/or the exogenous mitochondrial DNA population. Each possibility represents a separate embodiment of the invention.

According to certain embodiments, the enrichment of the stem cells with exogenous mitochondria may be determined by conventional assays that are recognized in the art. In certain embodiments, the level of mitochondrial enrichment in the mitochondrially-enriched human target cells is determined by (i) the levels of host/endogenous mitochondrial DNA and exogenous mitochondrial DNA; (ii) the level of mitochondrial proteins selected from the group consisting of citrate synthase (CS), cytochrome C oxidase (COX1), succinate dehydrogenase complex flavoprotein subunit A (SDHA) and any combination thereof; (iii) the level of CS activity; or (iv) any combination of (i), (ii) and (iii). Each possibility represents a separate embodiment of the invention.

In certain embodiments, the level of mitochondrial enrichment in the mitochondrially-enriched human stem cells is determined by at least one of: (i) the levels of host mitochondrial DNA and exogenous mitochondrial DNA in case of allogeneic mitochondria; (ii) the level of citrate synthase activity; (iii) the level of succinate dehydrogenase complex flavoprotein subunit A (SDHA) or cytochrome C oxidase (COX1); (iv) the rate of oxygen (O₂) consumption; (v) the rate of adenosine triphosphate (ATP) production or (vi) any combination thereof. Each possibility represents a separate embodiment of the present invention. Methods for measuring these various parameters are well known in the art.

In some embodiments, enrichment of the stem cells with exogenous human mitochondria comprises washing the mitochondrially-enriched target cells after incubation of the human stem cells with said isolated exogenous human exogenous mitochondria. This step provides mitochondrially-enriched target cells substantially devoid of cell debris or mitochondrial membrane remnants and mitochondria that did not enter the stem cells. In some embodiments, washing comprises centrifugation of the mitochondrially-enriched target cells after incubation of the human target cells with said isolated exogenous human mitochondria. According to some embodiments, the methods produce mitochondrially-enriched human stem cells that are separated from free mitochondria, i.e., mitochondria that did not enter the stem cells, or other cell debris and the pharmaceutical compositions contain mitochondrially-enriched human stem cells that are separated from free mitochondria. According to some embodiments, the methods produce and the pharmaceutical compositions contain mitochondrially-enriched human stem cells that do not comprise a detectable amount of free mitochondria.

In certain embodiments, the method described above further include concentrating the target cells and the isolated exogenous mitochondria before or during incubation and/or contacting. In certain embodiments, the method described above further includes centrifugation of target cells and isolated exogenous mitochondria before, during or after incubation or contacting. In some embodiments, the methods described above in various embodiments thereof include a single centrifugation step before, during or after incubation of the target cells with the isolated mitochondria.

In certain embodiments, the centrifugation speed is about 7,000 g or 8,000 g. According to further embodiments, the centrifugation is at a speed between 300 g-8000 g; 500 g-8000 g; 1000 g-8000 g; 300 g-5000 g; 2000 g-4000 g; 2500 g-8500 g; 3000 g-8000 g; 4000 g-8000 g; 5,000-10,000 g 7000 g-8000 g or above 2500 g. In some embodiments, centrifugation is performed for a time ranging from about 2 minutes to 30 minutes; 3 minutes to 25 minutes; 5 minutes to 20 minutes; or 8 minutes to 15 minutes.

In some embodiments, centrifugation is performed in a temperature ranging from about 2 to 6° C.; 4 to 37° C.; 4 to 10° C. or 16-30° C. In specific embodiments, centrifugation is performed at 4° C. In some embodiments, the methods described above in various embodiments thereof include a single centrifugation before, during or after incubation of the target cells with the isolated exogenous mitochondria, followed by resting the cells at a temperature lower than 30° C. In some embodiments, the conditions allowing the isolated exogenous mitochondria to enter the human target cells include a single centrifugation before, during or after incubation of the target cells with the isolated mitochondria, followed by resting the cells at a temperature ranging between 16 to 28° C.

In some embodiments, the methods generate and/or the pharmaceutical compositions contain mitochondrially-enriched stem cells at a concentration of at least 104 to 2×10⁸; 5×10⁵ to 1.5×10⁷; or 5×10⁵ to 4×10⁷ mitochondrially-enriched target cells per kilogram bodyweight of the subject. In some embodiments, the methods generate and/or pharmaceutical compositions contain mitochondrially-enriched target cells at a concentration of at least 10⁶ to 10⁷ mitochondrially-enriched human stem cells per kilogram bodyweight of the patient. In other embodiments, the methods generate and/or the pharmaceutical compositions contain mitochondrially enriched target cells at a concentration of at least 10⁵ or at least 10⁶ mitochondrially-enriched human stem cells per kilogram bodyweight of the patient. In some embodiments, the methods generate and/or the pharmaceutical compositions contain mitochondrially-enriched stem cells at a concentration of a total of at least 5×10⁵ up to 5×10⁹ mitochondrially-enriched target cells. In some embodiments, the methods generate and/or the pharmaceutical compositions contain mitochondrially-enriched target cells at a concentration of a total of at least 10⁶ up to 10⁹ mitochondrially-enriched target cells. In other embodiments, the methods generate and/or the pharmaceutical compositions comprises a total of at least 2×106 up to 5×10⁸ mitochondrially-enriched target cells.

In certain embodiments, target cells are fresh. In certain embodiments, the target cells are frozen and then thawed prior to incubation. In certain embodiments, the isolated exogenous mitochondria are fresh. In certain embodiments, the isolated exogenous mitochondria are frozen and then thawed prior to incubation. In certain embodiments, the mitochondrially-enriched target cells are fresh. In certain embodiments, the mitochondrially-enriched target cells are frozen and then thawed prior to administration.

In certain embodiments, the mitochondria are not frozen. In further embodiments, the isolated mitochondria are frozen, then stored and thawed prior to use. In further embodiments the mitochondrially-enriched target cells are used without freezing and storage. In yet further embodiments, the mitochondrially-enriched target cells are used after freezing, storage and thawing. Methods suitable for freezing and thawing of cell preparations in order to preserve viability are well known in the art.

As used herein, the term “freeze-thaw cycle” refers to freezing of the isolated exogenous mitochondria to a temperature below 0° C., maintaining the mitochondria in a temperature below 0° C. for a defined period of time and thawing the isolated mitochondria to room temperature or body temperature or any temperature above 0° C. which enables treatment of the target cells with the isolated mitochondria. The term “room temperature”, as used herein typically refers to a temperature of between 18° C. and 25° C. The term “body temperature”, as used herein, refers to a temperature of between 35.5° C. and 37.5° C., preferably 37° C.

In another embodiment, the mitochondria that have undergone a freeze-thaw cycle were frozen at a temperature of −20° C. or lower; −4° C. or lower; or −70° C. or lower. According to another embodiment, freezing of the mitochondria is gradual. According to some embodiment, freezing of mitochondria is through flash-freezing. As used herein, the term “flash-freezing” refers to rapidly freezing the mitochondria by subjecting them to cryogenic temperatures.

In another embodiment, the mitochondria that underwent a freeze-thaw cycle were frozen for at least 30 minutes prior to thawing. According to another embodiment, the freeze-thaw cycle comprises freezing the isolated exogenous mitochondria for at least 30, 60, 90, 120, 180, 210 minutes prior to thawing. Each possibility represents a separate embodiment of the present invention. In another embodiment, the isolated exogenous mitochondria that have undergone a freeze-thaw cycle were frozen for at least 1, 2, 3, 4, 5, 6, 7, 8, 9, 10, 24, 48, 72, 96, or 120 hours prior to thawing. In another embodiment, the isolated exogenous mitochondria that have undergone a freeze-thaw cycle were frozen for at least 4, 5, 6, 7, 30, 60, 120, 365 days prior to thawing. According to another embodiment, the freeze-thaw cycle comprises freezing the isolated exogenous mitochondria for at least 1, 2, 3 weeks prior to thawing. According to another embodiment, the freeze-thaw cycle comprises freezing the isolated exogenous mitochondria for at least 1, 2, 3, 4, 5, 6 months prior to thawing. Each possibility represents a separate embodiment of the present invention. According to another embodiment, the oxygen consumption of the isolated exogenous mitochondria after the freeze-thaw cycle is equal or higher than the oxygen consumption of the exogenous mitochondria prior to the freeze-thaw cycle.

According to certain embodiment, thawing is at room temperature. In another embodiment, thawing is at body temperature. According to another embodiment, thawing is at a temperature which enables administering the mitochondria according to the methods of the invention. According to another embodiment, thawing is performed gradually.

In certain embodiments, the method described above further includes a preceding step of administering to the subject afflicted with a disease or disorder or the donor an agent which induces mobilization of bone-marrow cells to peripheral blood.

In certain embodiments, the agent which induces mobilization of bone-marrow cells/stem cells produced in the bone marrow to peripheral blood is selected from the group consisting of granulocyte-colony stimulating factor (G-CSF), granulocyte-macrophage colony-stimulating factor (GM-CSF), 1,1′-[1,4-Phenylenebis(methylene)]bis [1,4,8,11-tetraazacyclotetradecane] (Plerixafor, CAS number 155148-31-5), CXCR4 inhibitors, a salt thereof, and any combination thereof. Each possibility represents a separate embodiment of the invention.

In certain embodiments, the method described above further includes isolating the stem cells from the peripheral blood of the subject afflicted with a disease or disorder and/or the donor. The term “isolating from the peripheral blood” as used herein refers to the isolation of stem cells from other constituents of the blood.

During apheresis, the blood of a subject or donor is passed through an apparatus that separates out one particular constituent and returns the remainder to the circulation. It is thus a medical procedure which is performed outside the body. In certain embodiments, the isolation is performed by apheresis.

In certain embodiments, the target cells, which may be stem cells, are obtained from a subject afflicted with a disease or disorder or a donor, and the target cells have (i) a normal rate of oxygen (O₂) consumption; (ii) a normal content or activity level of citrate synthase; (iii) a normal rate of adenosine triphosphate (ATP) production; or (iv) any combination of (i), (ii) and (iii).

In certain embodiments, the target cells, which may be stem cells, are obtained from a subject afflicted with a disease or disorder or a donor, and the target cells have (i) a decreased rate of oxygen (O₂) consumption; (ii) a decreased content or activity level of citrate synthase; (iii) a decreased rate of adenosine triphosphate (ATP) production; or (iv) any combination of (i), (ii) and (iii), as compared to a subject not afflicted with a disease or disorder.

In certain embodiments, the mitochondria-enriched target cells have (i) an increased rate of oxygen (O₂) consumption; (ii) an increased content or activity level of citrate synthase; (iii) an increased rate of adenosine triphosphate (ATP) production; (iv) an increased mitochondrial DNA content(v) a lower level of heteroplasmy or (vi) any combination of (i), (ii), (iii) (iv) and (v) as compared to the target cells.

The term “increased rate of oxygen (O₂) consumption” as used herein refers to a rate of oxygen (O₂) consumption which is detectably higher than the rate of oxygen (O₂) consumption prior to mitochondria enrichment.

The term “increased content or activity level of citrate synthase” as used herein refers to a content or activity level of citrate synthase which is detectably higher than the content value or activity level of citrate synthase prior to mitochondria enrichment.

The term “increased rate of adenosine triphosphate (ATP) production” as used herein refers to a rate of adenosine triphosphate (ATP) production which is detectably higher than the rate of adenosine triphosphate (ATP) production prior to mitochondria enrichment.

According to some aspects, the present invention provides a method of treating a disease or disorders or a symptom thereof in a human subject in need of such treatment, the method comprising the step of administering a pharmaceutical composition comprising a plurality of mitochondrially-enriched target cells to the subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intravenous, intraperitoneal, intraarterial, and intramuscular. The phrases “parenteral administration” and “administered parenterally” as used herein means modes of administration other than enteral and topical administration. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The present invention further provides, in another aspect, a pharmaceutical composition comprising a plurality of mitochondrially-enriched target cells as described above. In certain embodiments, the pharmaceutical composition described above is for use in a method of treating certain symptoms in a human subject having a disorder.

As used herein, “pharmaceutical composition” refers to a formulation comprising an active ingredient, and optionally a pharmaceutically acceptable carrier, diluent or excipient. The term “active ingredient” can interchangeably refer to an “effective ingredient”, and is meant to refer to any agent that is capable of inducing a sought-after effect upon administration. Examples of active ingredient include, but are not limited to, chemical compound, drug, therapeutic agent, small molecule, etc.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

In certain embodiments, the symptom is selected from the group consisting of impaired walking capability, impaired motor skills, impaired language skills, impaired memory, weight loss, cachexia, low blood alkaline phosphatase levels, low blood magnesium levels, high blood creatinine levels, low blood bicarbonate levels, low blood base excess levels, high urine glucose/creatinine ratios, high urine chloride/creatinine ratios, high urine sodium/creatinine ratios, high blood lactate levels, high urine magnesium/creatinine ratios, high urine potassium/creatinine ratios, high urine calcium/creatinine ratios, glucosuria, magnesuria, high blood urea levels, low C-Peptide level, high HbAlC level, hypoparathyroidism, ptosis, hearing loss, cardiac conduction disorder, low ATP content and oxygen consumption in lymphocytes, mood disorders including bipolar disorder, obsessive compulsive disorder, depressive disorders, as well as personality disorders. Each possibility represents a separate embodiment of the present invention. It should be understood that defining symptoms as “high” and “low” correspond to “detectably higher than normal” and “detectably lower than normal”, respectively, wherein the normal level is the corresponding level in a plurality of subjects not afflicted with a mitochondrial disease.

In certain embodiments, the enriched stem cells are administered to a specific tissue or organ. In certain embodiments, the enriched stem cells have at least 10⁴ mitochondrially-enriched target cells.

In certain embodiments, the mitochondrially-enriched target cells are administered by parenteral administration. In certain embodiments, the pharmaceutical composition is administered by systemic administration; intravenous injection or intravenous infusion. In certain embodiments, the enriched stem cells have at least 10⁵ mitochondrially-enriched target cells. In certain embodiments, the mitochondrially-enriched target cells have about at least 10⁴; at least 10⁴ to at least 10⁸; at least 10⁶ to at least 10⁸; at least 10⁵ to at least 2×10⁷; at least 10⁶ to at least 5×10⁶; or at least 10⁵ mitochondrially-enriched target cells.

In certain embodiments, the mitochondrially-enriched target cells, which may be stem cells, have at least one of (i) an increased mitochondrial DNA content compared to the mitochondrial DNA content in the target cells prior to mitochondrial enrichment; (ii) an increased rate of oxygen (O₂) consumption compared to the rate of oxygen (O₂) consumption in target cells prior to mitochondrial enrichment; (iii) an increased content or activity level of citrate synthase compared to the content or activity level of citrate synthase in target cells prior to mitochondrial enrichment; (iv) an increased rate of adenosine triphosphate (ATP) production compared to the rate of adenosine triphosphate (ATP) production in target cells prior to mitochondrial enrichment; (v) a lower level of heteroplasmy; or any combination of (i), (ii), (iii) (iv) and (v).

In certain embodiments, the total amount of mitochondrial proteins in the isolated exogenous mitochondria, is between 10%-80%, 20-70%, 40-70%, 20-40%, or 20-30% of the total amount of cellular proteins. Each possibility represents a separate embodiment of the present invention. In certain embodiments, the total amount of mitochondrial proteins in the isolated exogenous mitochondria, is between 20%-80% of the total amount of cellular proteins within the sample. In certain embodiments, the total amount of mitochondrial proteins in the isolated exogenous mitochondria, is between 20%-80% of the combined weight of the mitochondria and other sub-cellular fractions. In other embodiments, the total amount of mitochondrial proteins in the isolated exogenous mitochondria, is above 80% of the combined weight of the mitochondria and other sub-cellular fractions.

In some embodiments, the methods described above in various embodiments thereof further includes expanding the target cells by culturing said stem cells in a proliferation medium capable of expanding the target cells. In other embodiments, the method further comprises expanding the mitochondrially-enriched target cells by culturing said cells in a culture or proliferation medium capable of expanding target cells. As used throughout this application, the term “culture or proliferation medium” is a fluid medium such as cell culture media, cell growth media, buffer which provides sustenance to the cells.

In certain embodiments, the target cells are allogeneic to the subject afflicted with the disorder. The term “allogeneic to the subject” refers to the stem cells or mitochondria being HLA matched to the cells of the patient or at least partially HLA matched. According to certain embodiments, the donor is matched to the subject according to identification of a specific mitochondrial DNA haplogroup. In certain embodiments, the subject is the source of stem cells and/or mitochondria.

The term “HLA-matched” as used herein refers to the desire that the subject and the donor of the target cells be as closely HLA-matched as possible, at least to the degree in which the subject does not develop an acute immune response against the target cells of the donor. The prevention and/or therapy of such an immune response may be achieved with or without acute or chronic use of immune-suppressors. In certain embodiments, the stem cells from the donor are HLA-matched to the patient to a degree wherein the patient does not reject the stem cells.

In certain embodiment, the patient is further treated by an immunosuppressive therapy to prevent immune rejection of the stem cells graft.

The term “haplogroup” as used herein refers to a genetic population group of people who share a common ancestor on the matriline. Mitochondrial haplogroup is determined by sequencing.

In certain embodiments the mitochondria are from identical haplogroups. In other embodiments the mitochondria are from different haplogroups.

In certain embodiments, the method described above further includes a preceding step of administering to the subject a pre-transplant conditioning agent prior to the administration of the pharmaceutical composition. The term “pre-transplant conditioning agent” as used herein refers to any agent capable of killing bone-marrow cells within the bone-marrow of a human subject. In certain embodiments, the pre-transplant conditioning agent is Busulfan.

According to certain embodiments, the isolated mitochondria are isolated from a donor selected from a specific mitochondria haplogroup, in accordance with the disorder of the subject.

Lymphocyte deficiency is a condition where a subject as an abnormally low level of lymphocytes. A subject can suffer from a T lymphocytopenia, B pymphocytopenia or NK lymphocytipenia. Lymphocyte deficiency is associated with corticosteroid use; viral, microbial and fungal infections; malnutrition; excessive physical exercise; systemic lupus erythematosus; rheumatoid arthritis; sarcoidosis and multiple sclerosis.

Lymphocyte deficiency-related disease or disorder is any disease or disorder wherein a subject has abnormally low levels of lymphocytes. Examples of a lymphosyte deficiency-related disease or disorder include viral, microbial and fungal infections; systemic lupus erythematosus; rheumatoid arthritis; sarcoidosis and multiple sclerosis.

In one embodiment, the present invention provides a method for diminishing debilitating effects of a lymphocyte deficiency -related disease or diseases in a subject including incubating of hematopoietic stem cells (HSCs) with isolated exogenous mitochondria under conditions allowing the isolated exogenous mitochondria to enter the HSCs and administering the HSCs from (a) to the subject. In certain aspects, the HSCs are autologous or allogenic stem cells.

In an additional aspect, the exogenous mitochondria are isolated from a donor. In a further aspect, the exogenous mitochondria have undergone at least one freeze-thaw cycle. In various aspects, the HSCs are washed prior to administration to the subject.

Prior to bone marrow or hematopoietic stem cell transplantation subjects may go through a conditioning process to eliminate underlying disease and prevent rejection of the new cells. Conditioning regimens including the administration of chemotherapeutic agents and/or total body irradiation.

As used here in the term “conditioned subject” refers to a subject who is going to receive a bone marrow or HSC transplant and who has undergone a conditioning treatment involving the administration of chemotherapeutic agents and/or total body irradiation.

As used here in the term “non-condition subject” refers to a subject who is going to receive a bone marrow or HSC transplant and who has not undergone a conditioning treatment involving the administration of chemotherapeutic agents and/or total body irradiation.

In an additional aspect, the present invention provides a method for improving hematopoietic stem cell (HSC) transplantation in a subject including incubating of hematopoietic stem cells (HSCs) with isolated exogenous mitochondria under conditions allowing the exogenous mitochondria to enter the HSCs; and administering the HSCs to the subject. In certain aspects, the HSCs are autologous or allogenic stem cells. In an additional aspect, the exogenous mitochondria are isolated from a donor. In a further aspect, the exogenous mitochondria have undergone at least one freeze-thaw cycle. In various aspects, the HSCs are washed prior to administration to the subject.

In one embodiment the present invention provides a method of treating a disease or disorder by producing mitochondrially-enriched cells by contacting cells with exogenous mitochondria under conditions allowing the exogenous mitochondria to enter the cells; transducing the mitochondrially-enriched cell with a viral vector with a gene of interest; and administering the mitochondrially-enriched transduced cells to a subject. In one embodiment the present invention provides a method of treating a disease or disorder by transducing cells with a viral vector with a gene of interest; producing mitochondrially-enriched cells by contacting the transduced cells with exogenous mitochondria under conditions allowing the exogenous mitochondria to enter the cells; and administering the mitochondrially-enriched transduced cells to a subject. In one aspect, the cells are stem cells. In certain aspects, the cells are hematopoietic stem cells (HSC) or immunodeficient cells. In some aspects, the viral vector is an adeno-associated virus (AAV) vector or a lentivirus vector. In an additional aspect, the administration of the mitochondrially-enriched transduced cell increases the number of B cells compared with non-augmented cells. In certain aspects, the B cells are pre-B or pro-B cells. In a further aspect, the administration of the mitochondrially-enriched transduced cells increases the number of IgM positive cells compared with non-augmented cells. In one embodiment, mitochondrial enrichment increases the number of transduced cells.

A gene therapy technique is based on the transplantation of genetically modified autologous hematopoietic stem cells (HSCs). Gene therapy uses genes to treat or prevent disease. The most common form of gene therapy involves inserting a normal gene to replace an abnormal gene. Other approaches include swapping an abnormal gene for a normal one, repairing an abnormal gene, and altering the degree to which a gene is turned on or off. Stem cell gene therapy is based on the genetic modification of a relatively small number of stem cells. These persist long-term in the body by undergoing self-renewal and generate large numbers of genetically “corrected” progeny. HSCs are particularly attractive targets for gene therapy since their genetic modification will be passed to all the blood cell lineages as they differentiate.

Efficient long-term gene modification of HSCs and their progeny requires a technology which permits stable integration of the corrective DNA into the genome, without affecting HSC function. Accordingly, the use of integrating recombinant viral systems such as y-retroviruses, lentiviruses and spumaviruses has dominated this field (Chang, A. H. et al. (2007) Mol. Ther. 15: 445-456). Therapeutic benefits have already been achieved in y-retrovirus-based clinical trials for Adenosine Deaminase Severe Combined Immunodeficiency (ADA-SCID; Aiuti, A. et al. (2009) N. Engl. J. Med. 360: 447-458), X-linked Severe Combined Immunodeficiency (SCID-X1; Hacein-Bey-Abina, S. et al. (2010) N. Engl. J. Med. 363: 355-364) and Wiskott-Aldrich syndrome (WAS; Bortug, K. et al. (2010) N. Engl. J. Med. 363: 1918-1927). In addition, lentiviruses have been employed as delivery vehicles in the treatment of X-linked adrenoleukodystrophy (ALD; Cartier, N. et al. (2009) Science 326: 818-823), and for metachromatic leukodystrophy (MLD; Biffi, A. et al. (2013) Science 341: 1233158) and WAS (Aiuti, A. et al. (2013) Science 341 : 1233151).

A vector can be an integrating or non-integrating vector, referring to the ability of the vector to integrate the expression cassette and/or transgene into a genome of a cell. Either an integrating vector or a non-integrating vector can be used to deliver an expression cassette containing a gene operably linked to a regulatory element. Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs);

episomal vectors; transposons (e.g., PiggyBac); and (b) viral vectors such as retroviral vectors, lentiviral vectors, adenoviral vectors, and AAV vectors. Viruses have several advantages for delivery of nucleic acids, including high infectivity and/or tropism for certain target cells or tissues. In some cases, a virus is used to deliver a nucleic acid molecule or expression cassette comprising one or more regulatory elements, as described herein, operably linked to a gene.

Expression vectors can include regulatory elements to control transcription of the polynucleotide of interest. Non-limiting examples of regulatory elements include promoter, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Vectors also can include other elements. For example, a vector can include a nucleic acid that encodes a signal peptide such that the encoded polypeptide is directed to a particular cellular location (e.g., a signal secretion sequence to cause the protein to be secreted by the cell) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyl transferase (XGPRT). Such markers are useful for selecting stable transformants in culture. Regulatory sequences can generally be derived from mammalian, microbial, viral or insect genes. The ability to replicate in a host, usually conferred by an origin of replication, and a selection gene to facilitate recognition of transformants. Those of skill in the art can select a suitable regulatory region to be included in such a vector.

A vector can be a genomic integrated vector, or “integrated vector,” which can become integrated into the chromosomal DNA of the host cell; or an episomal vector, e.g., a nucleic acid capable of extra-chromosomal replication. Vectors capable of directing the expression of genes to which they are operatively linked are referred to herein as “expression vectors.” Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

The term “AAV” is an abbreviation for adeno-associated virus, and can be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAVS, AAV6, AAV7, AAV8, AAV9, AAV10, AAV11, AAV 12, rhlO, and hybrids thereof, avian AAV, bovine AAV, canine AAV, equine AAV, primate AAV, non-primate AAV, and ovine AAV.

The use of “lentiviral vector” in gene therapy refers to a method by which genes can be inserted, modified, or deleted in organisms using lentivirus. Lentivirus are a family of viruses which infect by inserting DNA into their host cells' genome. Many such viruses have been the basis of research using viruses in gene therapy, but the lentivirus is unique in its ability to infect non-dividing cells, and therefore has a wider range of potential applications. Lentiviruses can become endogenous (ERV), integrating their genome into the host germline genome, so that the virus is henceforth inherited by the host's descendants. To be effective in gene therapy, there must be insertion, alteration and/or removal of host cell genes. To do this scientists use the lentivirus' mechanisms of infection to achieve a desired outcome to gene therapy. Non-limiting examples or lentivirus that can be used for gene therapy include those derived from bovine immunodeficiency virus, caprine arthritis encephalitis virus, equine infectious anemia virus, feline immunodeficiency virus, Human immunodeficiency virus 1, Human immunodeficiency virus 2, Jembrana disease virus, puma lentivirus, simian immunodeficiency virus or Visna-maedi virus.

In addition to the use of retro- and lentiviral-based vectors, vectors derived from other viruses, such as adenoviruses and adeno-associated viruses (AAV), may also be utilized for the modification of hematopoietic stem and progenitor cells.

As known to one of skill in the art, gene modification can be achieved by targeted gene editing by employing site-specific endonucleases to induce a double-stranded break (DSB) in the DNA (e.g. Zinc finger nucleases (ZFNs), Transcription activator like effector nucleases (TALENs), clustered regularly interspaced short palindromic repeats (CRISPR).

In another embodiment, gene therapy can be used to enhance gene expression.

According to some embodiments, augmented transduced cells provide increase proliferation compared to nonaugmented transduced cells. According to some embodiments, augmented transduced cells have increase differentiation compared to nonaugmented transduced cells. In another aspect, transduced cells that have been augmented with mitochondria have increased gene expression compared to non-augmented transduced cells. According to some embodiments, augmented transduced cells have increased number of cells expressing the transgene compared to non-augmented transduced cells.

In a further embodiment, the present invention provides a method for improving hematopoietic stem cells (HSC) transplantation in a subject including incubating of hematopoietic stem cells (HSCs) with isolated exogenous mitochondria under conditions allowing the exogenous mitochondria to enter the HSCs; and administering the HSCs to the subject. In one aspect, the HSC are genetically modified. In an additional aspect, the subject has a disease or disorder selected from Primary immune deficiency (e.g. Wiskott-Aldrich Syndrome, leukocyte adhesion deficiency, X-linked hyper IgM syndrome, X-linked lymphoproliferative disease, X-linked Agammaglobulinemia, X-linked severe combined immune deficiency, chronic granulomatous disease), Hemoglobinopathies (e.g. sickle cell disease, beta-thalassemia), Storage and metabolic disorders (e.g. Gaucher Disease and other lipidoses, mucopolysaccharidoses (I-VII), X-linked Adrenoleukodystrophy, metachromatic leukodystrophy, osteopetrosis), Congenital cytopenias and stem cell defects (e.g. Fanconi anemia, Schwachman-Diamond Syndrome, Kostmann's Syndrome). In a further aspect, the Lysosomal Storage Disorders is Gaucher disease type I. In certain aspects, the HSCs are autologous or allogenic stem cells. In an additional aspect, the exogenous mitochondria are isolated from a donor. In a further aspect, the exogenous mitochondria have undergone at least one freeze-thaw cycle. In various aspects, the HSCs are washed prior to administration to the subject.

In one embodiment, the present invention provides a method for treating immunodeficiency or immune related diseases in a subject by incubating of hematopoietic stem cells (HSCs) with exogenous mitochondria under conditions allowing the exogenous mitochondria to enter the HSCs; and administering the HSCs to the subject. In certain aspects, the HSCs are autologous or allogenic stem cells. In an additional aspect, the exogenous mitochondria are isolated from a donor. In a further aspect, the exogenous mitochondria have undergone at least one freeze-thaw cycle. In one aspect, the HSCs are expanded in vitro. In an additional aspect, the HSCs have undergone at least one freeze-thaw cycle. In a further aspect, the HSCs have undergone at least one freeze thaw cycle prior to or following in vitro expansion. In certain aspects, the HSCs have undergone at least one freeze thaw cycle prior to or following incubation with the exogenous mitochondria. In an additional aspect, the conditions allowing the exogenous mitochondria to enter the target cells may include incubating the target cells with the exogenous mitochondria at a ratio of about 0.088-176 mU citrate synthase (CS) activity per 10⁶ cells.

As used herein the terms “treatment” and “treating” refer to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

Immune related diseases are diseases that affect the immune system. Such diseases and disorders include autoimmune diseases and immunodeficiency diseases and disorders. An autoimmune disease is an illness that causes the immune system to produce antibodies that attack normal body tissues. Examples of autoimmune disease include Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyclinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barr, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, and myasthenia gravis.

Immunodeficiency diseases and disorders impair the immune system's ability to defend the body against foreign or abnormal cells that invade or attack it (such as bacteria, viruses, fungi, and cancer cells). As a result, unusual bacterial, viral, or fungal infections or lymphomas or other cancers may develop. There are two types of immunodeficiency disorders primary and secondary. Primary immunodeficiency disorders are usually present at birth and are genetic disorders that are usually hereditary. They typically become evident during infancy or childhood. However, some primary immunodeficiency disorders (such as common variable immunodeficiency) are not recognized until adulthood. Examples of common primary immunodeficiency disorders include Wiscott-Aldrich syndrome, Severe combined immunodeficiency disease (SCID), DiGeorge syndrome, Ataxia-telangectasia, Chronic granulomatous disease, Transient hypogammaglobulinemia of infancy, Agammaglobulinemia, Complement deficiencies and Selective IgA deficiency.

Secondary immunodeficiency disorders generally develop later in life and often result from use of certain drugs or from another disorder, such as diabetes or human immunodeficiency virus (HIV) infection. They are more common than primary immunodeficiency disorders. Examples of common secondary immunodeficiency disorders include HIV, leukemia, lymphoma and multiple myeloma.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Example 1

### Transplatation of Enriched Stem Cells in to Mouse Model

Mitochondria were isolated from the blood of a heathy human donor. The mitochondria were frozen in −80° C. CD34+ cells were isolated from frozen and thawed umbilical cord blood cells (UBCs) of a subject having Pearson syndrome. The subject was diagnosed with a deletion of 4,977 nucleotides in positions 8,470-13,447 in the mtDNA. The mitochondria were thawed and then the subject CD34+ cells were incubated with 0.88 mU of human mitochondria per 1×10⁶ cells for 22 hours. Subsequently, the media was removed, and cells were washed and resuspended in 4.5% HSA. Using sequence analysis, augmentation was verified by identifying the presence of the human mitochondria in the cells. The augmented cells were then I.V. injected to a 3 week old NSGS mouse (50K cells per mouse).

3 groups of mice were tested: MAT group: NSGS mice transplanted with human augmented CD34+ cells, 50K cells/150 ul. Control group: NSGS mice transplanted with human CD34+ cells, 50K cells/150 ul. Naïve group: Naïve mouse.

TABLE 1

| Group | Number of mice |
|---|---|
| MAT: UBC+ MITO | 6 |
| augmentation | |
| Control: UBC | 7 |
| Naïve | 1 |

2 months after transplantation:

3 mice from the MAT group and 3 mice from the control group, were sacrificed. Peripheral blood (PB) was drawn, and bone marrow (BM) cells were isolated from femur and tibia bones. To determine the copy number of human endogenous and exogenous mitochondrial DNA (mtDNA), DNA was isolated from the BM and PB and analyzed using dPCR.

To determine the differentiation of the CD34+ cells to subpopulations of hematopoietic cells, peripheral blood and BM samples were analyzed in flow cytometry. First, the % of human CD45+ cells was determined since the CD45 antigen is present on all human leucocytes, including lymphocytes, monocytes, granulocytes, eosinophils, and basophils in peripheral blood.

Subsequently, the CD45+ cells were further divided into the subpopulations of CD45+CD33+ (Myeloid lineage), CD45+CD3+ (T cells), CD45+CD19+ (B cells), CD45+CD14+ (Monocytes).

Furthermore, flow cytometry analysis was utilized to determine the % of mCD45 cells vs. hCD45 cells.

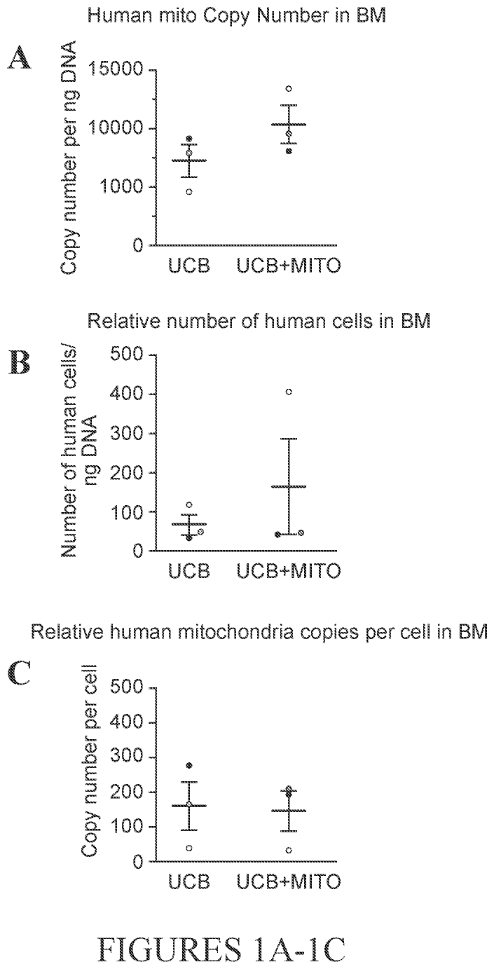

Results BM: 2 months after treatment the results for the BM between the MAT group and the control group are shown in FIG. 1.

TABLE 2

| | | CD45+ | | | |
|---|---|---|---|---|---|
| | CD45 | % | % | % | % |
| Sample | Count | hCD3 | hCD14 | hCD19 | hCD33 |
| Nsgs calib 00014769 | 49.05 | 0.15 | 9.58 | 4.97 | 52.9 |
| BM MITO 2 | | | | | |
| Nsgs calib 00014769 | 5.12 | 0.58 | 47.8 | 6.57 | 93.9 |
| BM MITO 3 | | | | | |
| Nsgs calib 00014769 | 11.8 | 1.31 | 36 | 6.86 | 79.1 |
| BM MITO 1 | | | | | |
| Nsgs calib 00014769 | 6.91 | 0.2 | 25.8 | 6.55 | 69.7 |
| BM UCB 1 | | | | | |
| Nsgs calib 00014769 | 24.6 | 0.45 | 14.5 | 7.81 | 62.3 |
| BM UCB 2 | | | | | |
| Nsgs calib 00014769 | 7.44 | 0.63 | 44.3 | 6.01 | 96.1 |
| BM UCB 3 | | | | | |
| Nsgs calib 00014769 | 0.071 | | | | |
| UNST BM 3 | | | | | |

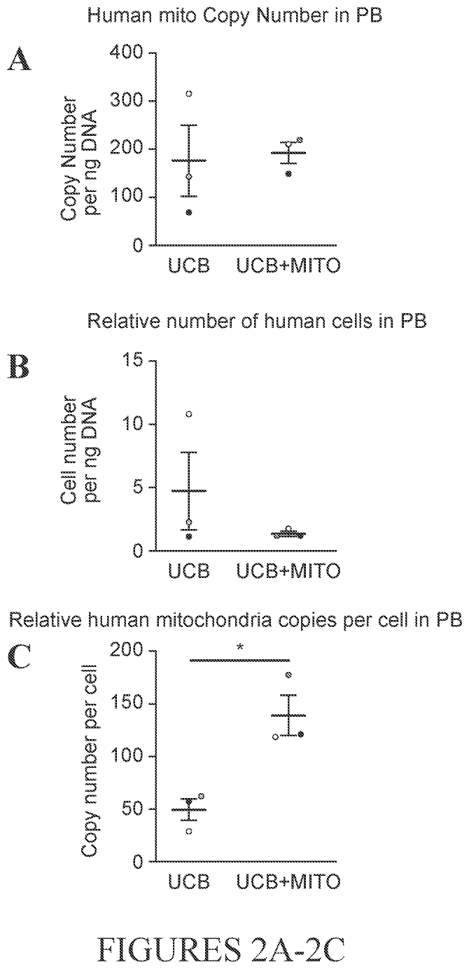

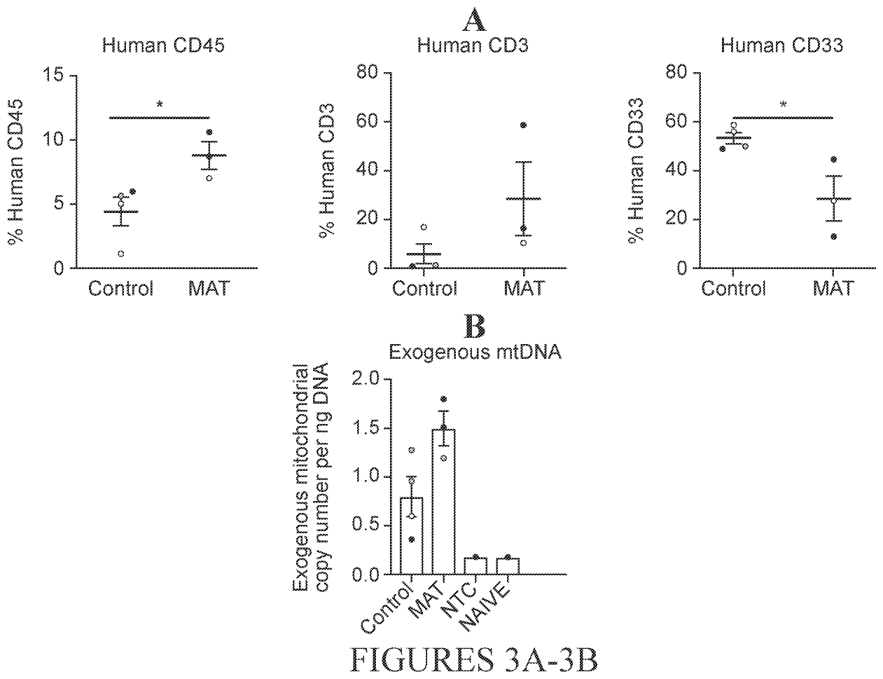

Results PB: PB dPCR: The results for human mitochondria copy number, the human cell number and mtDNA copies in the PB, between the control group and the MAT group are shown in FIG. 2. A significant increase in total (exogenous+endogenous) mtDNA copies per cell was observed in the peripheral blood.

6 months after transplantation:

The mice from each group and the naïve mouse were sacrificed. To measure the levels of endogenous and exogenous human mtDNA, DNA was isolated from the BM and peripheral blood and analyzed using digital PCR (dPCR).

Results BM: BM dPCR exhibited the exogenous mitochondria copy number per ng DNA for the control group (mice comprising non-augmented human cells), MAT group (mice transplanted with mitochondria-augmented human cells), NTC (“no template”) , naïve group (no treatment). Since the SNP assay used is based on a single nucleotide variation that distinguishes between two alleles of endogenous and exogenous human mtDNA (G/A at position 10831 of the mtDNA), the selectivity towards a specific allele is not perfect and therefore false positive results were also apparent in the control group. Nonetheless, BM cells derived from MAT mice had significantly more exogenous mitochondria copy number per ng DNA indicating increased engraftment of the MAT cells and possibly increased cell proliferation. (FIG. 3, right bar graph).

BM Flow cytometry results: 6 months after transplantation BM cells derived from MAT mice comprised significantly higher levels of CD45+ human cells compared to BM cells derived from the control mice. The increase in the CD45+ cells in MAT mice indicate increase engraftment, improved homing, increased cell proliferation, increased cell viability, or decrease cell apoptosis. The human CD45+ cells were further analyzed for their differentiation to sub populations. CD45+ cells subpopulations included CD3+ (T-cells), CD14+ (macrophages), CD19+ (B cells), CD33+ cells (myeloid cells).

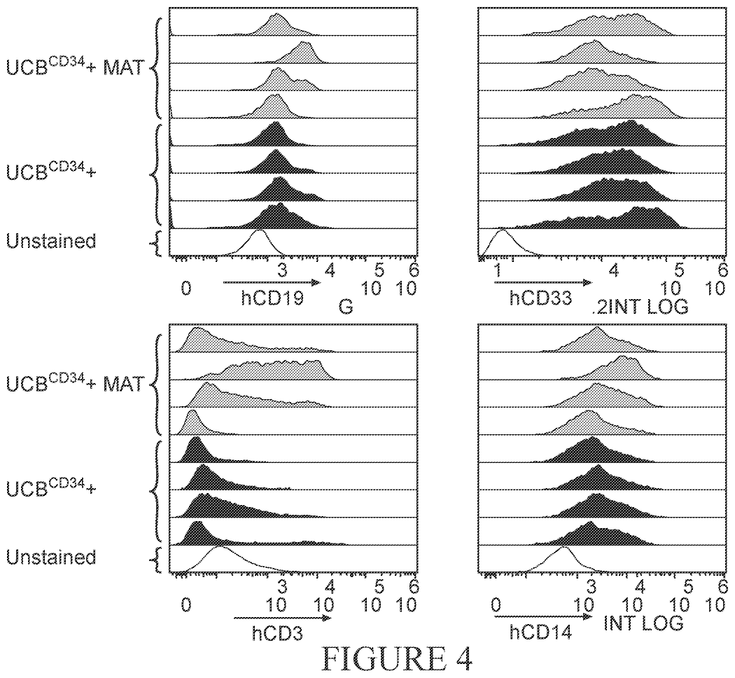

MAT mice had a different pattern of cell differentiation compared to the control. The MAT mice had a higher % of CD3+ cells (T-Cells) and a lower % of CD33+ cells and the control mice had a higher % of CD33+ cells and lower % of CD3+ cells, as seen in flow cytometry analysis. MAT mice also had a higher % of CD19+ cells compared to control mice. These results indicate a shift from myeloid cells to lymphoid cells (FIG. 4 and Table 3).

TABLE 3

| | | CD45+ | | |
|---|---|---|---|---|
| | CD45+ | % hCD33 | % hCD3 | % hCD19 |
| UCB | 7.92 | 44.9 | 16.3 | 0.15 |
| MAT4 | | | | |
| UCB | 8.49 | 13.1 | 58.7 | 0.34 |
| MAT5 | | | | |
| UCB | 7.07 | 28.1 | 10.4 | 0.17 |
| MAT6 | | | | |
| UCB4 | 5.66 | 50.2 | 1.51 | 0.095 |
| UCB5 | 6.01 | 49.3 | 0.52 | 5.55E−03 |
| UCB6 | 5 | 67.3 | 4.38 | 0.096 |
| UCB7 | 1.14 | 56.3 | 17.5 | 0.29 |

### Example 2

### Treating Lymphocyte Deficiency/Increasing Lyphocyte Population in a Subject

The steps of the method for diminishing debilitating effects of lymphocyte deficiency in a subject suffering from a lymphocyte deficiency-related disease or diseases are: (1) obtaining autologous or allogeneic hemopoietic stem cells; (2) isolating mitochondria from the cells of a donor. The isolation of the exogenous mitochondria can be performed prior to this process, storing the mitochondria frozen at −80° C. (at least) and defrosted prior to use; (3) incubation of HSCs with the isolated exogenous mitochondria; (4) washing the bone marrow cells; and (5) infusion of HSCs enriched with mitochondria to the subject. During the entire period, evaluating changes in the patient's blood counts and biochemical blood markers.

### Example 3

### Mitochondrial Augmentation Therapy for Imprving Bone Marrow Transplantation

To improve engraftment and therefore cell transplantation in conditioned or non-conditioned patients, the cells undergo mitochondrial augmentation prior to transplantation to the patient. A method to improve hematopoietic stem cell transplantation in a subject in need thereof comprises (1) obtaining hematopoietic stem cells (HSCs) from the subject or a donor; (2) isolating mitochondria from the cells of a donor (the isolation of the mitochondria can be performed prior to this process, storing the mitochondria frozen at −80° C. (at least) and defrosted prior to use); (3) incubating of HSCs with the isolated exogenous mitochondria; (4) washing the bone marrow cells; and (5) administering of HSCs enriched with mitochondria to the subject. The HSCs maybe autologous or allogeneic hematopoietic stem cells.

### Example 4

### Mitochondrial Augmentation Therapy for Improving Gene Therapy

Efficient long-term gene modification of HSCs and their progeny requires a technology which permits stable integration of the corrective DNA into the genome, without affecting HSC function. Accordingly, the use of integrating recombinant viral systems such as y-retroviruses, lentiviruses and spumaviruses has dominated this field (Chang, A. H. et al. (2007) Mol. Ther. 15: 445-456). Therapeutic benefits have already been achieved in y-retrovirus-based clinical trials for Adenosine Deaminase Severe Combined Immunodeficiency (ADA-SCID; Aiuti, A. et al. (2009) N. Engl. J. Med. 360: 447-458), X-linked Severe Combined Immunodeficiency (SCID-X1; Hacein-Bey-Abina, S. et al. (2010) N. Engl. J. Med. 363: 355-364) and Wiskott-Aldrich syndrome (WAS; Bortug, K. et al. (2010) N. Engl. J. Med. 363: 1918-1927). In addition, lentiviruses have been employed as delivery vehicles in the treatment of X-linked adrenoleukodystrophy (ALD; Cartier, N. et al. (2009) Science 326: 818-823), and for metachromatic leukodystrophy (MLD; Biffi, A. et al. (2013) Science 341: 1233158) and WAS (Aiuti, A. et al. (2013) Science 341 : 1233151).

The Clustered Regularly Interspersed Short Palindromic Repeats/CRISPR-associated (CRISPR/Cas) system, CRISPR/Cas system, is a powerful tool for rapid genome engineering in which a single guide RNA (sgRNA) containing a spacer sequence complementary to the targeted DNA sequence guides a DNA endonuclease enzyme (such as a Cas9 enzyme for example), to a genomic target. Upon binding, Cas9 creates a double-strand DNA break. DNA repair mechanisms, non-homologous end joining (NHEJ) or homologous recombination (HR), can be exploited to introduce gene insertions and deletions. CRISPR/Cas9 has been implemented in various species, such as Escherichia coli, S. cerevisiae, and mammalian cells. Other examples of DNA endonuclease enzymes include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1,Csb2, Csb3, Csx17, Csx14, Csx10, Csx16, CsaX, Csx3, Csx1, Csx15, Csf1, Csf2, Csf3 and Csf4.

The elements of CRISPR systems (e.g., direct repeats, homologous recombination editing templates, guide sequences, tracrRNA sequences, target sequences, priming sites, regulatory elements, and RNA-guided DNA endonucleases) are well known to those of skill in the art. That is, given a target sequence, one of skill in the art can design functional CRISPR elements specific for a particular target sequence. The methods described herein are not limited to the use of specific CRISPR elements.

In addition to the use of retro- and lentiviral-based vectors, vectors derived from other viruses, such as adenoviruses and adeno-associated viruses (AAV), may also be utilized for the modification of hematopoietic stem and progenitor cells.

To enhance the engraftment of cells intended to be use in gene therapy (e.g. Lysosomal Storage Disorders such as Gaucher disease type I Primary immune deficiencies), the cells undergo augmentation with isolated exogenous mitochondria. Cells may undergo augmentation prior to, during, or after genetically modifying the HSC for gene therapy. A method for enhancing engraftment of cells in a subject comprises (1) obtaining hematopoietic stem cells (HSCs) from the subject or a donor; (2) isolating mitochondria from the cells of a donor (the isolation of the mitochondria can be performed prior to this process, storing the mitochondria frozen at −80° C. (at least) and defrosted prior to use); (3) incubating of HSCs with the isolated exogenous mitochondria; (4) washing the bone marrow cells; and (5) administering of HSCs enriched with mitochondria to the subject. The HSCs maybe autologous or allogeneic hematopoietic stem cells.

### Example 5

### Continuous Transfer of Exogenous Mitochondria in Mitochondrial Dysfunction Mouse Model Following Mitochondrial Augmentation

To assess biodistribution and whether augmented HSPCs or their progeny are capable of transferring mitochondria to other cells in vivo in a diseased animal model, genetically labelled cells were injected into a mouse model of mitochondrial dysfunction (PolG mouse).

A new murine in vivo system was used which will help track where mitochondrially augmented bone marrow cells, or the mitochondria they harbor, home following intravenous infusion. ROSA^{nT-nG}/PhAM^{excised} mice mouse model was used in which all cell nuclei were labelled with red fluorescence (dTomato) and all mitochondria were labelled with green fluorescence (protein Cox8-Dendra), hereby called ‘red-green’ cells. Augmentation was performed with ‘green’ mitochondria isolated from the ROSA^{nT-nG}/PhAM^{excised} mice.

As distribution or persistence of HSPCs and exogenous mitochondria may be affected by organismal stress, the Polg mouse model was used in which a PolgD²⁵⁷A mutation impairs proofreading functions of the mitochondrial DNA polymerase, leading to accumulation of mtDNA mutations and deletions. The PolG mouse model was treated with augmented red— green HSPCs to assess in-vivo biodistribution of mitochondrially augmented cells and of their mitochondria.

Lineage negative cells (Lin) ubiquitously expressing red-labeled nucleus (dTomato) and green-labeled mitochondria (Dendra) derived from ROSAnT⁻nG/PhAM^{excised} mice were augmented with Dendra mitochondria isolated from liver cells of the ROSAnT⁻nG/PhAM^{excised} mice and injected to Polg mice dosed at age 25-28 weeks (males) and 30-35 weeks (females).

TABLE 4

| | Normalized fold | # Cells |
|---|---|---|
| | of COX-1 levels | injected/mouse |
| Lin - Females | 1.9 | 197 K |
| Lin - Males | 1.5 | 242 K |

Augmentation level was measured with protein level of mt-DNA-encoded cytochrome c oxidase 1 (COX-1) in cells after MAT. COX-1 values were normalized to non-treated cells and to cell number as assessed by janus green

In the recipient PolG mouse three cell types are potentially identifiable: 1) red-green cells, originating from infused cells or their progeny, 2) non-fluorescing cells, originating from the PolG recipient, and 3) green-only cells, providing evidence of mitochondrial transfer to recipient PolG cells.

At 12 hours, 1 week and 4.5 months post-injection, blood was drawn and BM was extracted and both PB and BM were analyzed by flow cytometry and confocal microscopy.

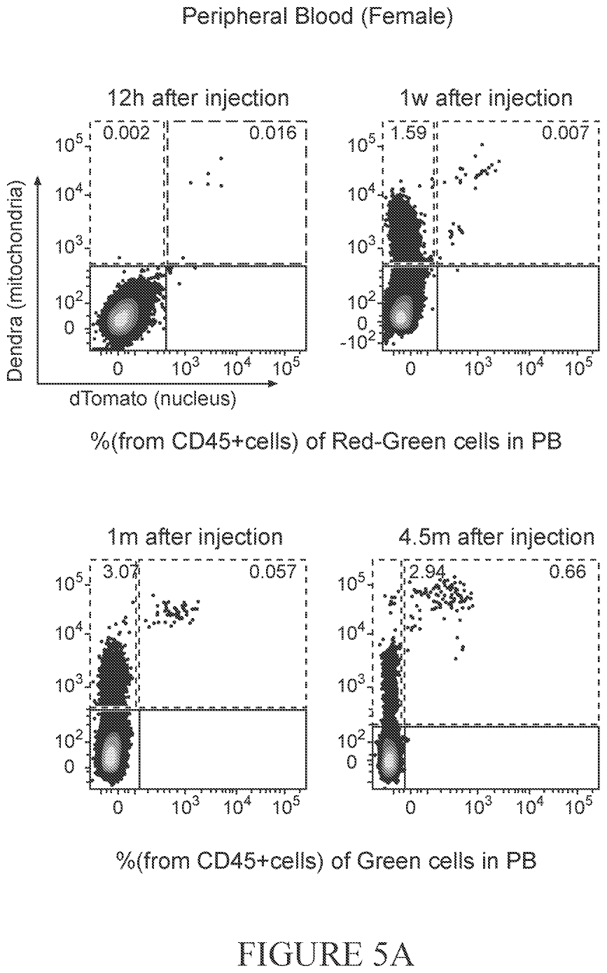

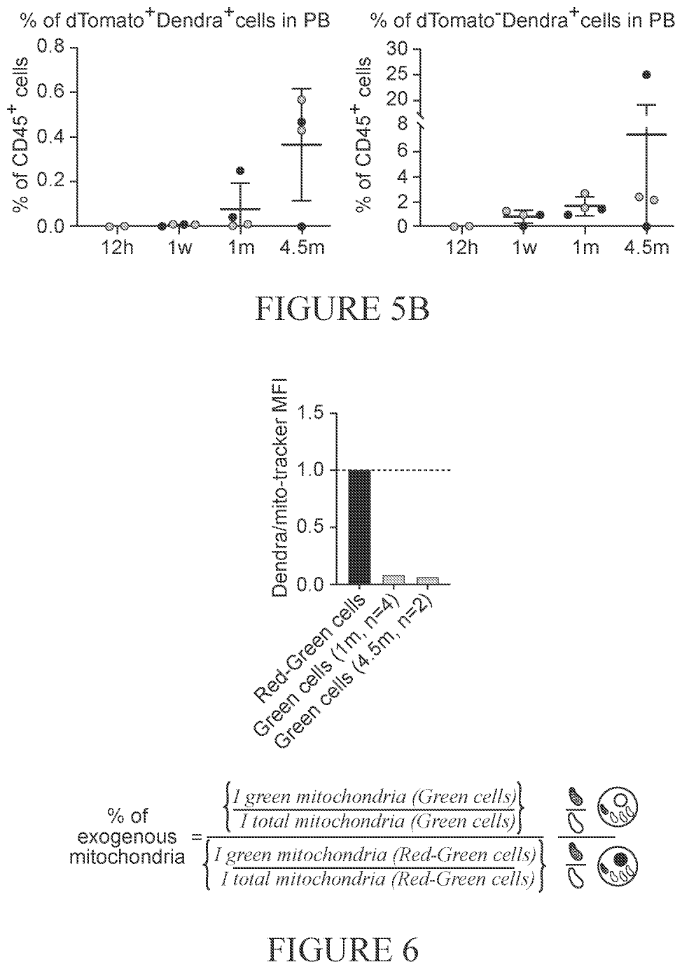

Long term persistence and continual transfer of exogenous mitochondria from infused HSPCs to endogenous cells in the peripheral blood was demonstrated. FIGS. 5A-B demonstrate the frequency of CD45⁺ cell subsets within the PB. The proportion of dTomato⁺Dendra⁺ proportions increased over the time course from 0.0015% at 12 h to 0.37% at 4.5m post-injection. Similarly, the proportions of dTomato⁻Dendra⁺ cells to which mitochondrial transfer occurred, increased from 0.0015% to 7.3%. As the mitochondrial Cox8-Dendra2 protein is only encoded by the ROSA^{nT-nG}/PhAM^{excised} nuclear genome, and therefore transient once passing to a Polg recipient cell, consistent presence of dTomato⁻Dendra⁺ cells in the peripheral blood is evidence of continual transfer of exogenous mitochondria which originated from the infused cells or their progeny.

To determine the relative levels of exogenous mitochondria that were transferred to recipient cells, peripheral blood cells were stained with MitoTracker Deep-red. The fold change of the ratio between exogenous to total mitochondria in the cells that received mitochondria (green cells) to the red-green cells was measured.

It was demonstrated that exogenous mitochondria were 8.4% at 1 month and 6.1% at 4.5 months of the total cell mitochondria content. (FIG. 6).

Cells that contain green mitochondria were also found in mouse kidney 4.5 months after MAT.

As green mitochondria were present in recipient PolG cells, this data suggests that there is persistence and continual transfer of the exogenous mitochondria. Importantly this is a lower limit for exogenous mitochondria content, as it is limited by the half-life of Cox8-Dendra protein.

### Example 6

### Mitochondria are Transferred to a Variety of Hematopoietic Cell Types

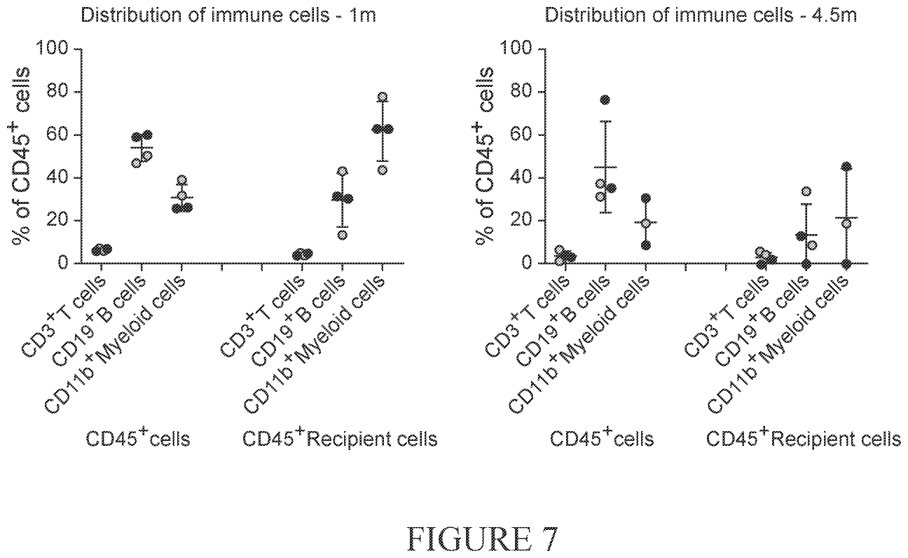

Using flow cytometry, hematopoietic cell types were tested to determine which cell types were the recipients of intracellular mitochondrial transfer. The distribution of the major immune cell subsets in PB was assessed in both total CD45⁺ and CD45⁺dTomato-Dendra⁺ populations at 1 m and 4.5 m time-points (FIG. 7).

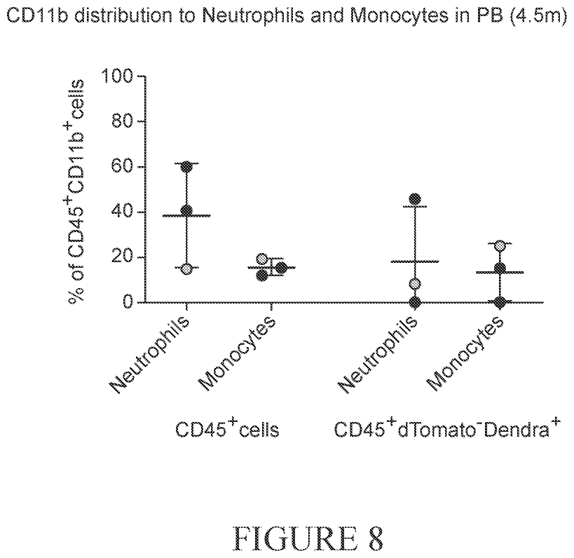

At the 4.5 m time point, the myeloid population uptaking exogenous mitochondria was further characterized, and demonstrated that both monocytes (Ly6C^{high}Ly6G⁻) and neutrophils (Ly6C⁺LY6G⁺) uptake mitochondria (FIG. 8).

The myeloid cell population preferentially uptook the exogenous mitochondria, but numerous cell types (including T cells and B cells) were all demonstrated to uptake mitochondria.

The data demonstrates that exogenous mitochondria originating from the infused HSPCs are continually transferred to various hematopoietic cell types present in the peripheral blood. Furthermore, evidence of cells harboring exogenous mitochondria in distant tissues suggests that exogenous mitochondria persist not only in immune cells but also in non-hematopoietic tissues.

In summary, it was demonstrated that mitochondria from infused HSPCs can be transferred to additional hematopoietic cells in the peripheral blood and in distal tissues over at least 4.5 months after MAT treatment.

### Example 7

### Assessment of Cell Function Following Mitochondrial Augmentation

It has been observed that CD11b⁺ myeloid cells in peripheral blood of PolG mice uptake exogenous mitochondria from dTomato⁺Dendra⁺ cells. Therefore, the activity level of CD11b⁺ myeloid cells comprising exogenous mitochondria is further assessed.

dTomato⁺Dendra⁺ lineage negative cells were augmented with Dendra2⁺ mitochondria isolated from the liver of the ROSA^{nT-nG}/PhAM^{excised} mice for 21 hours at room temperature and IV injected to PolG mice.

Peripheral blood (PB) is extracted from the untreated PolG mice and from treated mice at 1 week, 1 month, 3 months, and 4.5 months after treatment. The extracted PB is sorted for myeloid cells by FACS. Myeloid cells extracted from untreated or vehicle control treated mice are used as control in the following assays.

To assess differences in activity of myeloid cells of treated and control mice, RNAseq is conducted on the cells to evaluate myeloid cell transcriptomic changes. Similarly, RNASeq is conducted on specific myeloid cell subpopulations (neutrophils, eosinophils, monocytes, etc.) sorted by cell surface marker or by single cell RNASeq. In addition, myeloid cell or myeloid cell subpopulation activity level is assessed in cell-based assays including but not limited to cytokines expression, metabolic and mitochondrial activities including ATP level, CS activity, mitochondria membrane potential, or cell-specific functional activity assessments such as monocyte to macrophage differentiation, phagocytosis assays, response to inducers, and NETosis assays.

Effect of augmentation on myeloid cell subpopulations can further be evaluated with the assays described above using myeloid cells isolated from naive and mitochondrially augmented murine or human PBMCs.

It is expected that myeloid precursor cells that received mitochondria have increased proliferation, differentiation and/or activity, resulting in increased appearance of total blood myeloid cells and myeloid subpopulations. Additionally, blood myeloid cells will be primed by the mitochondria uptake after mitochondrial augmentation, resulting in a more robust function (e.g. neutrophil NETosis in response to pathogens or macrophage phagocytosis).

### Example 8

### Effect of Augmentation on Immune Deficient Cells and Gene Therapy

B cells developmental progression is guided by sequential events leading to assembly, expression, and signaling of the B cell antigen receptor (BCR). Heavy (H) and light (L) chain immunoglobulin genes are rearranged at the pro-B and pre-B stages (respectively), and complete surface IgM is expressed at the immature stage. Further developmental progression and maturation provide immune-competent collection of naïve B cells. Bruton's tyrosine kinase (BTK) gene is a critical component of the B-cell antigen receptor (BCR) signaling pathway and plays a crucial role in development, survival, and activation of B cells. The expression of BTK is not restricted to B cells, as BTK is also expressed in cells of myeloid lineage, including macrophages and neutrophils. X-linked deficient (Xid) mice carry a spontaneous mutation in the BTK gene. Having impaired B cell development, Xid mice are used as a model of human X-linked immunodeficiency disorder. For example, X-linked agammaglobulinemia (XLA), a genetic disorder resulting from mutations in the Bruton's tyrosine kinase (BTK) gene. These mutations lead to failure in B-lymphocyte maturation, low serum immunoglobulin levels and failure of specific antibody production as well as alternation of other immune signals.

These experiments determine the effect of augmentation on immune deficient cells and on gene therapy. This was done by testing B-cell development of immunodeficient cells that were augmented and transduced with a BTK gene.

Lin− cells were isolated from bone-marrow of Xid mice. Cells were either incubated with or without mitochondria derived from C57BL/6J mouse placenta (4.4 mU CS activity per 1×10⁶ cells) for 21 hours. After incubation, augmented and non-augmented cells, identical number of cells were either un-transduced or transduced with either a NTX109 or NTX101 lentiviral construct. Each construct comprised a cassette comprising BTK promoter-BTK transgene-A2-GFP (green fluorescent protein). Table 5 shows the groups tested.

TABLE 5

| Cells | MAT | Lentivector | Group name |
|---|---|---|---|
| WT | − | − | WT |
| (CBA/Ca) | | | |
| Xid Lin- | − | − | Non-augmented non-transduced Lin- |
| | − | NTX101 | Non-augmented NTX101 |
| | − | NTX109 | Non-augmented NTX109 |
| | + | − | Augmented non-transduced Lin- |
| | + | NTX101 | Augmented NTX101 |
| | + | NTX109 | Augmented NTX109 |

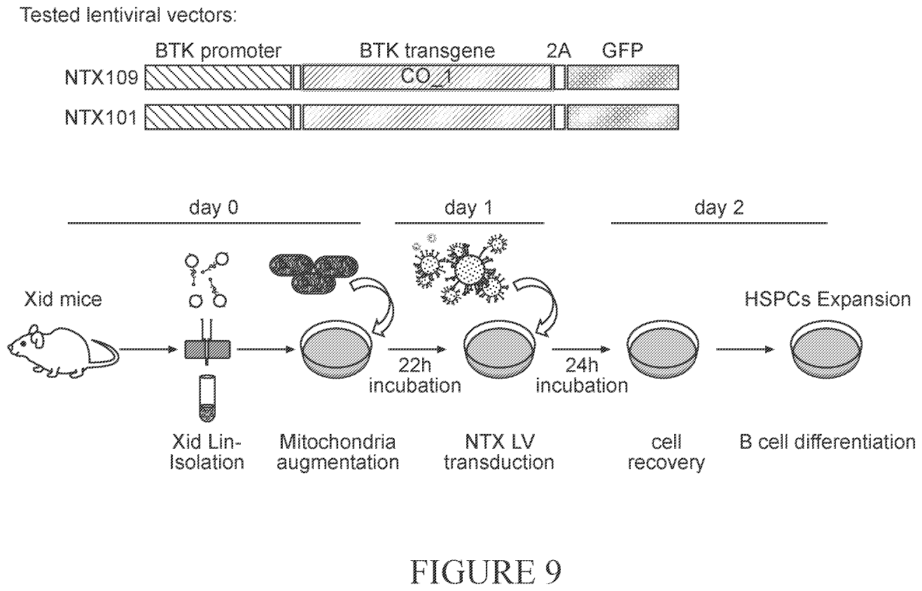

Twenty two hr after transduction, cells were washed and plated for recovery. On days 2 through 13 the cells were grown under conditions inducing B cell proliferation and differentiation, such as growing the cells in the presence of IL 7. On day 13, the cells were induced with LPS/CpG to undergo B-cell maturation, enabling the cells to differentiate into cells expressing B220 and IgM. On day 13 and on day 17, the cells were analyzed using flow cytometry to determine absolute cell number, and B-cells subpopulation. The experimental scheme is shown in FIG. 9.

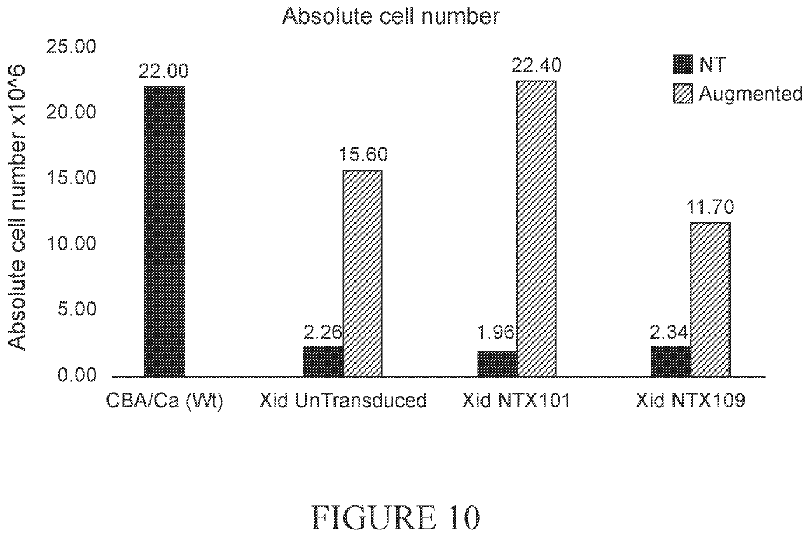

On day 13, a 11.4-fold and 5-fold increase in absolute cell number was observed in augmented NTX101 cells and augmented NTX109 cells, respectively, compared to the respective non-augmented NTX101 and the non-augmented NTX109 cells. Additionally, the cell number of augmented NTX109 cells corresponded to WT cell number (FIG. 10). A 6.9-fold increase in absolute cell number was observed in augmented non-transduced Lin− cells compared to Non-augmented non-transduced Lin− cells (FIG. 10)

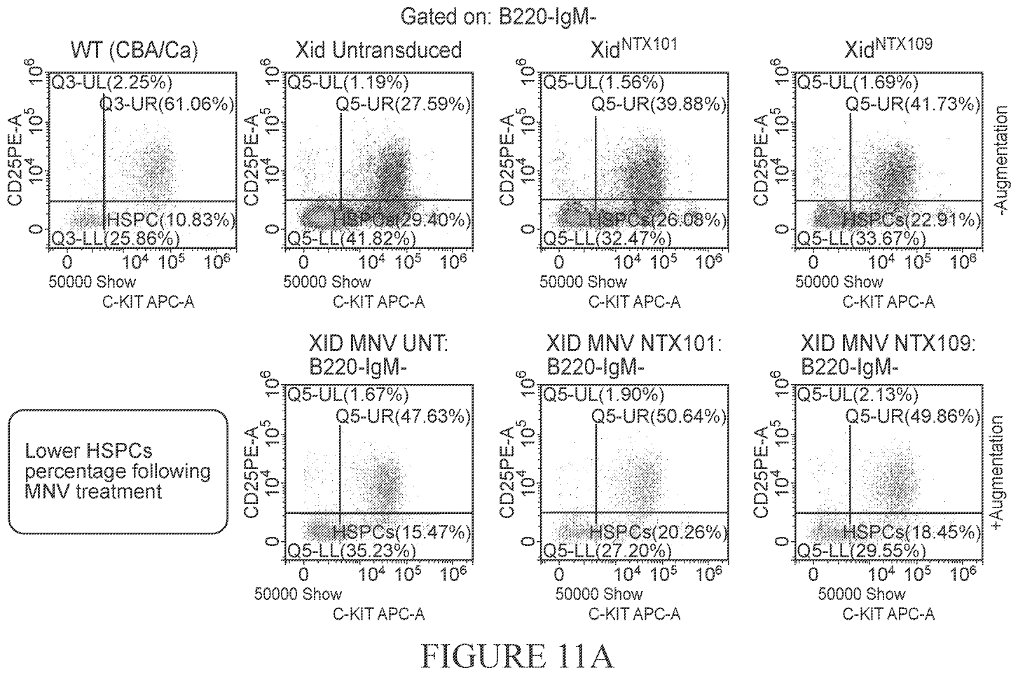

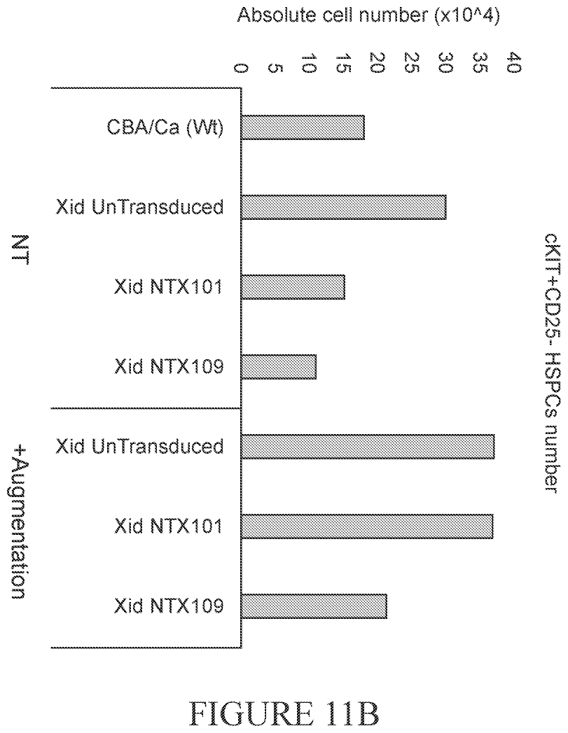

The HSPC population after was determined on day 13 (FIG. 11A-B). Augmented non-transduced Lin− cells had a 47% reduction in the HSPC population percentage compared to Non-augmented non-transduced Lin− cells. Non-augmented NTX101 cells exhibited an 11% reduction in HSPCs population percentage compared to Non-augmented non-transduced Lin− cells. Whereas Augmented NTX101 cells exhibited a 31% reduction in HSPCs population percentage compared to the Non-augmented non-transduced Lin− cells. Furthermore, augmented NTX101 cells exhibited a 22% reduction in HSPCs population percentage compared to Non-augmented NTX101 cells. Non-augmented NTX109 cells exhibited a 22% reduction in HSPCs population percentage compared to Non-augmented non-transduced Lin− cells. Whereas, a 37% reduction in HSPCs population percentage was observed in augmented NTX109 population compared to the Non-augmented non-transduced Lin− population. Additionally, a 19% reduction in HSPCs population percentage was observed in augmented NTX109 cells, compared to Non-augmented NTX109 cells. Additionally, augmented groups described in table 5 comprised significantly more HSPCs compared to their respective non augmented groups (FIG. 11B).

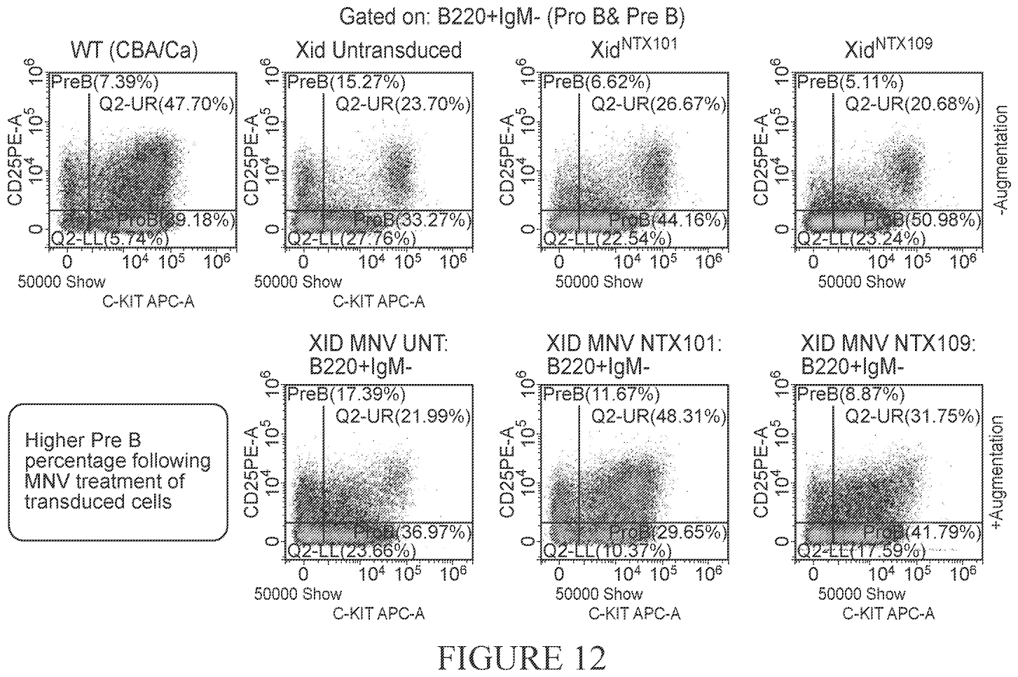

The B Cell population (pre-B and pro-B cells) was determined 13 days after augmentation. A higher pre-B cell population percentage was observed following augmentation. A 1.76 fold increase in percentage of pre-B cells was observed in augmented NTX101 cells compared to non-augmented NTX101 cells. A 1.73-fold increase in percentage of pre-B cells was observed in augmented NTX109 cells compared with non-augmented NTX109 cells. A 1.14-fold increase in percentage of pre-B cells was observed in augmented non-transduced Lin− cells, compared to non-augmented non-transduced cells (FIG. 12).

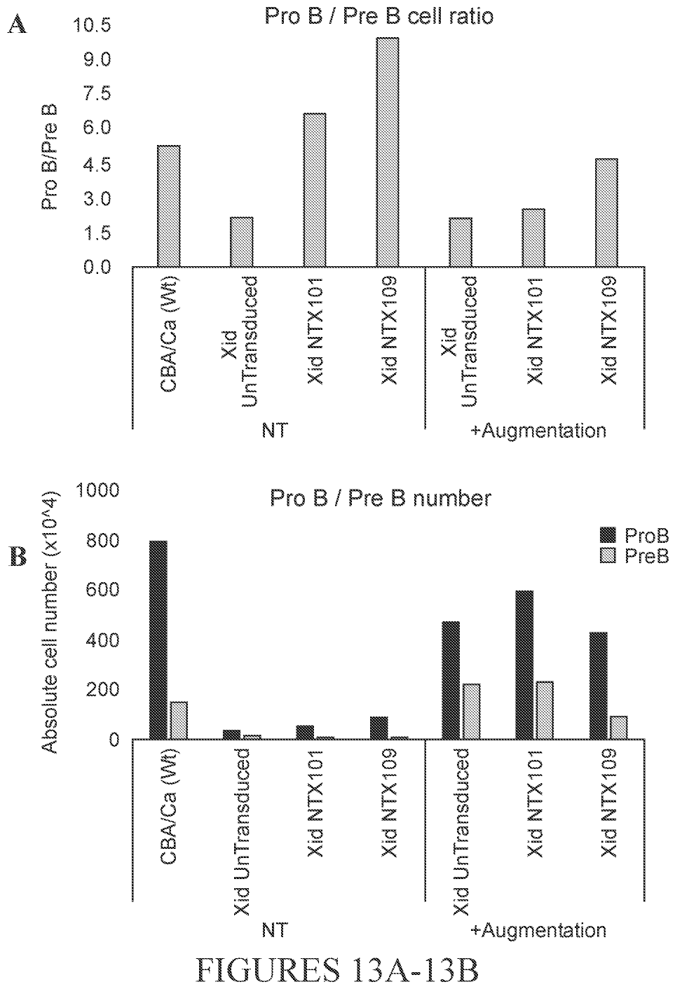

Immunophenotyping of the cells 13 after augmentation was performed (FIG. 13A-B). The Pro/Pre B cell ratio in augmented NTX101 cells was 2.54 whereas the Pro/Pre B cell ratio in non-augmented NTX101 cells was 6.6. Therefore, a 62% reduction in Pro B/Pre B cell ratio in augmented NTX101 cells compared to non-augmented NTX101 cells. A 53% reduction in Pro B/Pre B cell ratio in augmented NTX109 cells was observed compared to non-augmented NTX109 cells (Pro B/Pre B cell ratio: 4.7 vs. 9.9, respectively). The Pro B/Pre B cell ratio of augmented NTX109 cells was similar to the ratio found in WT cells. No significant change was observed in the Pro B/Pre B cell ratio of augmented non-transduced Lin− cells, compared to non-augmented non-transduced Lin− cells (ratio: 2.12 vs. 2.17). Additionally, augmented groups described in table 5 comprised significantly more Pre B cells and Pro B cells compared to their respective non augmented groups (FIG. 13B).

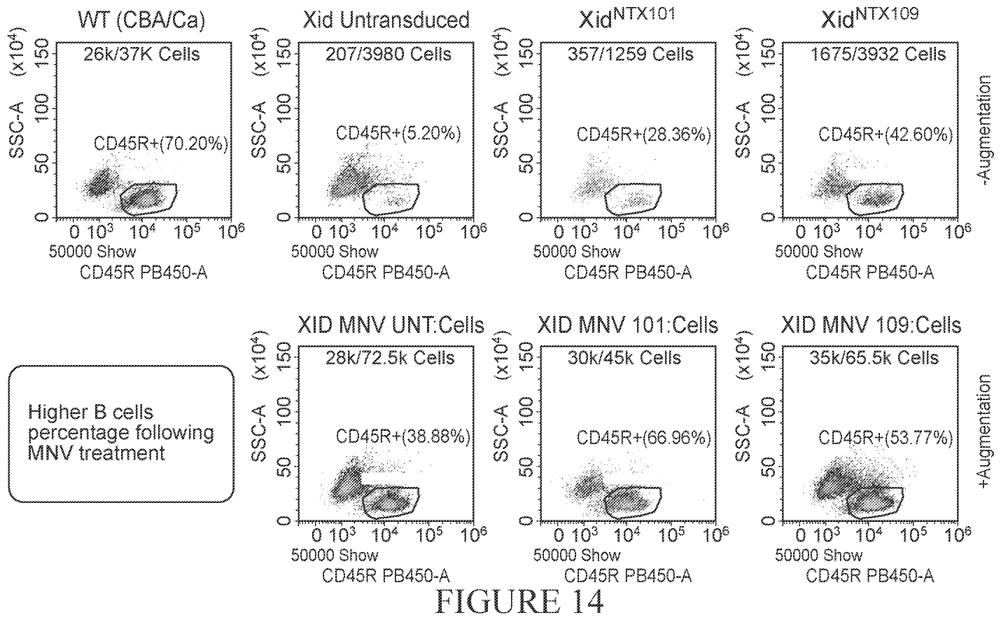

Cell number and B cell population was determined 17 days after augmentation (FIG. 14). Seventeen days following augmentation, augmented non-transduced cells exhibited an increase of 18-fold in cell number compared to non-augmented non-transduced Lin− cells (3,980 cells vs. 72,500 cells, respectively). Additionally, after 17 days, augmented non-transduced cells comprised 1.9-fold more cells compared to the WT (72,500 vs. 37,000 cells, respectively). The B cell population percentage of the augmented non-transduced cells was 7.4-fold higher than the B cell population of the non-augmented non-transduced cells, comprising 38.88% of the cell population, compared to 5.2% of the cell population in non-augmented non-transduced Lin− cells. Cells that were augmented and transduced exhibited 35-fold and 16-fold more cells compared to non-augmented transduced cells (NTX101 comprised 1,259 cells, whereas augmented NTX101 comprised 45,000, and NTX109 comprised 3,932 cells, whereas augmented NTX109 comprised 65,500 cells). A 3.3-fold increase was observed in B-cell population percentage of augmented NTX101 Lin− cells compared to non-augmented NTX101 cells (66.9% vs. 28.4%). A 1.26-fold increase was observed in B-cell population percentage of the cell population of augmented NTX109 cells compared to non-augmented NTX109 cells (53.8% vs. 42.6%).

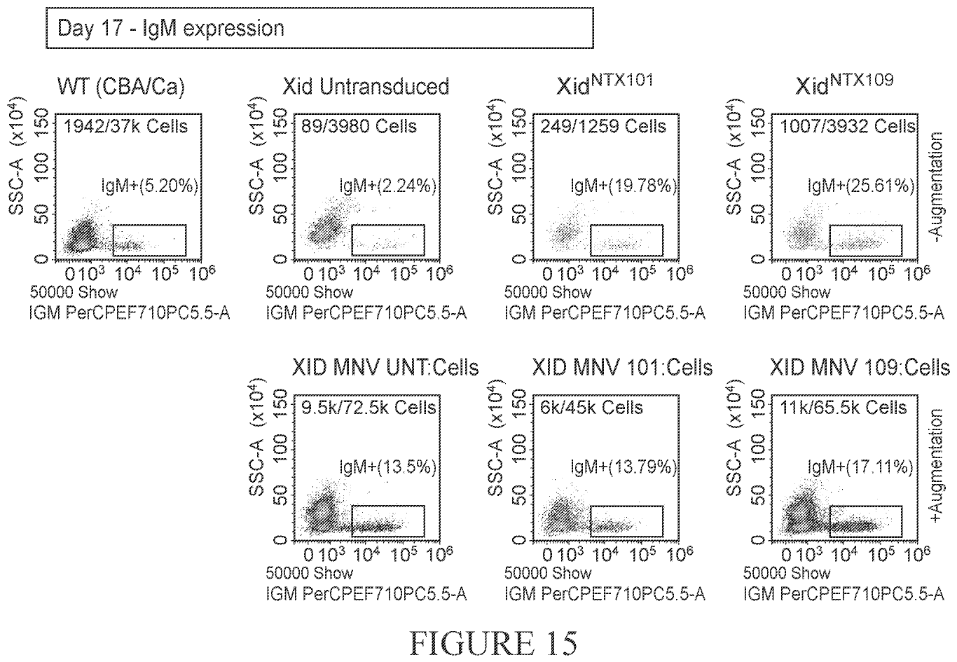

The effect of augmentation on IgM positive B-cells was determined seventeen days after augmentation (FIG. 15). Transduced cell did not show an increase in IgM positive percentage in cell populations after augmentation. IgM positive B-cells comprised 13.7% of the augmented NTX101 population compared to 19.7% of IgM positive cells of the non-augmented NTX101. For NXT109, 17.11% of cell population of the augmented NTX109 cells was IgM positive cells compared 25.61% of the cells of non-augmented NTX109 population. However, augmented non-transduced cells exhibited a 5.8-fold increase in percent of IgM positive B-cell population compared to non-augmented non-transduced Lin− cells (13.05% vs. 2.24%, respectively). The percentage of IgM positive cell population in augmented cells was 2.5-fold higher than IgM positive B-cells percentage in WT control mice (CBA) derived Lin− cells (13.05% vs. 5.20%, respectively). While IgM positive B cells relative portion of the cell population (i.e. the percentage of cells) was lower in augmented transduced cells compared to non-augmented transduced cells, the absolute number of total cells and therefore absolute number of IgM positive B cells, was significantly higher in cells that underwent augmentation.

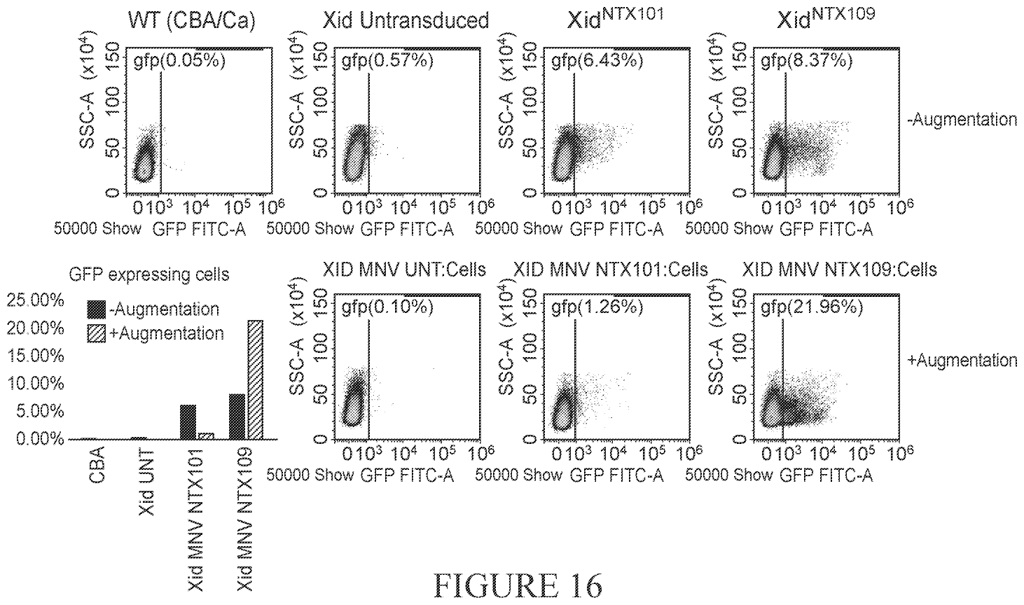

Gene expression was determined thirteen days after augmentation (FIG. 16). An identical cell number was extracted from each group of cells during the course of B-cell differentiation and analyzed for GFP expression using flow cytometry. 2.62-fold increase in percentage of cells expressing BTK, as reflected by GFP, was observed in augmented NTX109 cells, compared with non-augmented NTX109 cells (FIG. 16) indicating increased transduction of augmented NTX109. An increased GFP expression was not observed in augmented NTX101 cells compared to non-augmented NTX101 cells.

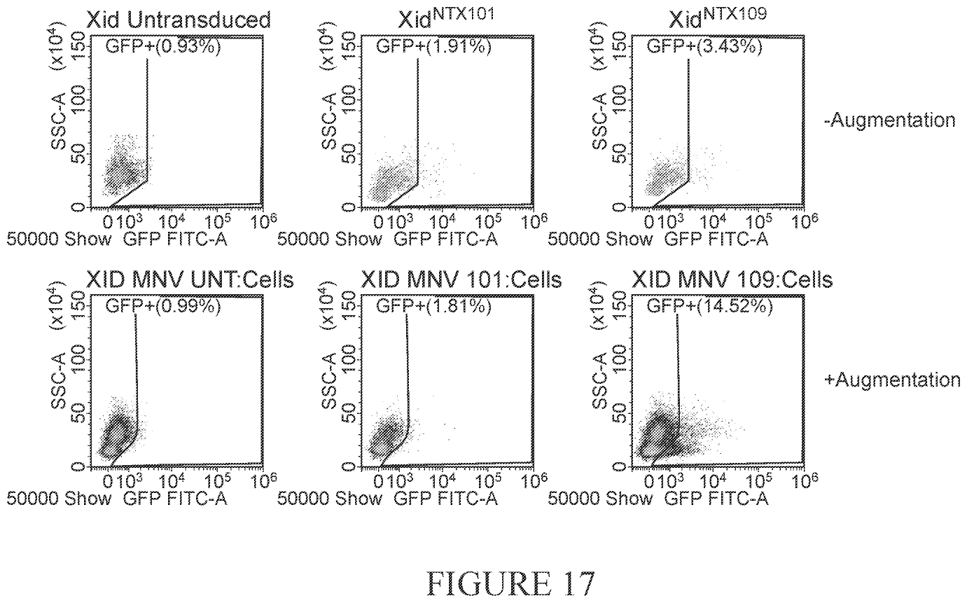

Gene expression was determined thirteen days after augmentation (FIG. 17). A 4.2-fold increase in BTK expression in augmented NTX109 cells compared to non-augmented NTX109 cells (14.52% vs. 3.43%, respectively) was observed.

Augmented Xid Lin− cells (transduced and non-transduced) exhibited increased cell proliferation, as measured by absolute number of cells, compared to non-augmented cells or compared to WT cells. Augmented Xid Lin− cells (transduced and non-transduced) exhibited a decrease in HSPCs percent of population. Augmented Xid Lin− cells (transduced and non-transduced) exhibited an increase in B-cell percentage of population and an increase in absolute cell number compared to non-augmented cells indicating enhanced B cell differentiation. The percentage of IgM positive cells was higher in augmented non-transduced cells compared to non-augmented non-transduced cells. The absolute number of total cells and therefore absolute number of IgM positive B cells, was significantly higher in all cell groups that underwent augmentation compared to non-augmented groups. Augmented cells transduced with a NTX109 vector exhibited increase % of BTK-expressing cells compared with non-augmented NTX109 cells, suggesting a beneficial effect of augmentation in restoring BTK expression in Xid Lin− cells.

### Example 9

### Effect of Augmentaitn on Gene Therapy

The effect of transplanting lethally irradiated Xid mice with augmented or non-augmented Xid Lin− cells transduced with lentivector (Xid^{pTC9}) or WT Lin⁻ cell (Xid^{CBA/Ca}) was assessed.

Xid mice were transplanted with augmented (MNV) or non-augmented (NT) Xid Lin− cells transduced with pTC9 lentivector comprising a GFP transgene under the PGK constitutive promotor. A parallel in vitro characterization of the transduced cells was performed to evaluate the expression levels of the transgene in the cellular product by measuring relative vector copy number (VCN) and protein expression of GFP compared to non-transduced Xid and WT Lin− cells.

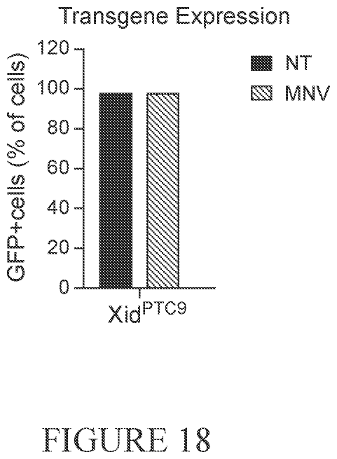

Relative VCN demonstrated comparable genomic integration events in all tested conditions. Transgene expression (percentage of GFP positive cells) was evident in all transduced cells (both augmented and control) (FIG. 18).

Blood was collected six weeks following transplantation of transduced Xid Lin− cells to xid mice, and transgene expression was assessed (N=5 mice for NT PtC9, N=4 mice for MNV PtC9) using flow cytometry.

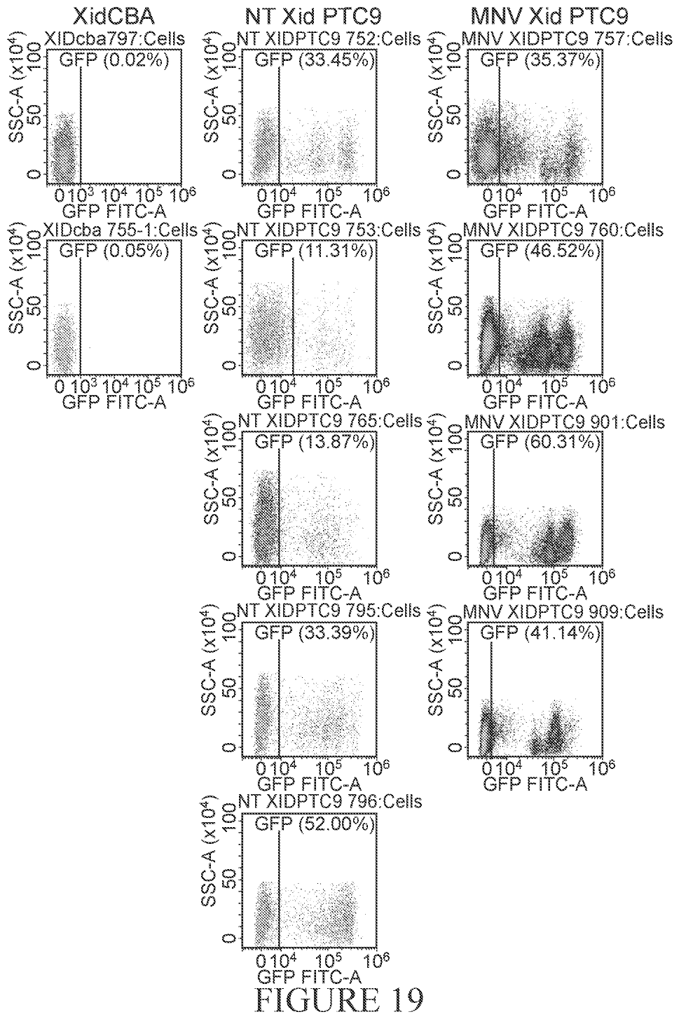

While there was variation between animals with respect to percent of cells expressing GFP, augmentation raised the minimum, maximum, and average percent of cells expressing GFP (FIG. 19). Table 6 shows the percent of cells expressing GFP in each transgenic and WT animal tested.

TABLE 6

| NT | MNV |
|---|---|
| PtC9 | PtC9 |
| 33.45 | 35.37 |
| 11.31 | 46.52 |
| 13.87 | 60.31 |
| 33.39 | 41.14 |
| 52 | |

Although the invention has been described with reference to the above examples, it will be understood that modifications and variations are encompassed within the spirit and scope of the invention. Accordingly, the invention is limited only by the following claims.

1. A method of increasing levels of leukocyte cells in a subject comprising:
(a) obtaining target cells from a subject;
(b) obtaining exogenous mitochondria from a donor cell;
c) producing mitochondrially-enriched target cells by contacting the target cells with the exogenous mitochondria under conditions allowing the exogenous mitochondria to enter the target cells; and
(d) administering the mitochondrially-enriched target cells to the subject,
wherein the mitochondrial content of the mitochondrially-enriched target cells is detectably higher than the mitochondrial content of the target cells, thereby increasing the levels of leukocyte cells in a subject.

2. The method of claim 1, wherein the target cells are selected from the group consisting of: pluripotent stem cells, embryonic stem cells, induced pluripotent stem cells, mesenchymal stem cells, hematopoietic stem cells, hematopoietic progenitor cells, common myeloid progenitor cells, common lymphoid progenitor cells, CD34+ cells, and any combination thereof.

3. The method of claim 1, wherein the target cells are CD34+ cells.

4. The method of claim 1, wherein the target cells are obtained from whole blood, blood fractions, peripheral blood, PBMC, serum, plasma, adipose tissue, placenta, oral mucosa, blood, umbilical cord blood or bone marrow.

5. The method of claim 1, wherein the subject has a disease or disorder.

6. The method of claim 5, wherein the disease or disorder is selected from the group consisting of: age related disorders, cancer, muscle diseases and disorders, glycogen-storage diseases and disorders, vascular endothelium disorder or diseases, brain disorder or brain disease, placental disorder or placental disease, thymus disorder or thymus disease, autoimmune diseases, renal disease or renal disorder, primary mitochondrial disease, pancreas disorder or pancreas disease, prostate disorder or prostate disease, kidney disorder or kidney disease, blood disorder or blood disease, heart disease or heart disorder, skin disorder or skin disease, immune and inflammatory diseases and disorders, bone disease or bone disorder, gastro-intestinal disease or gastro-intestinal disorder, eye disease or eye disorder and infection.

7. The method of claim 1, wherein the exogenous mitochondria are isolated or partially purified frozen-thawed human mitochondria.

8. The method of claim 1, wherein the exogenous mitochondria constitute at least 1% of the total mitochondria content in the mitochondrially-enriched target cells.

9. The method of claim 1, wherein the exogenous mitochondria are derived from a human cell or tissue selected from the group consisting of placenta, placental cells grown in culture, blood cells, and stem cell.

10. The method of claim 1, wherein the mitochondrial content of the mitochondrially-enriched target cells is determined by an assay selected from the group consisting of: content of at least one mitochondrial protein selected from SDHA and COX1; activity level of citrate synthase; rate of oxygen (O₂) consumption; rate of adenosine triphosphate production; mitochondrial DNA content, level of heteroplasmy and any combination thereof

11. The method of claim 1, wherein the administration of the mitochondrially-enriched target cells is by intravenous, intraperitoneal, intraarterial or intramuscular administration.

12. The method of claim 1, wherein between at least 5×10⁵ to 5×10⁹ mitochondrially-enriched target cells are administered to the subject.

13. The method of claim 1, wherein the mitochondrially-enriched target cells have:
(a) an increased content of at least one mitochondrial protein selected from SDHA and COX1;
(b) an increased rate of oxygen (O₂) consumption;
(c) an increased activity level of citrate synthase;
(d) an increased rate of adenosine triphosphate (ATP) production;
(e) an increased mitochondrial DNA content;
(f) a lower heteroplasmy level; or
(g) any combination thereof,
as compared to target cells prior to mitochondrial enrichment.

14. The method of claim 1, further comprising adding a pharmaceutically acceptable carrier to the mitochondrially-enriched target cells prior to administration to the subject.

15. The method of claim 1, wherein the conditions allowing the exogenous mitochondria to enter the target cells comprise incubating the target cells with the exogenous mitochondria at a ratio of about 0.088-176 mU citrate synthase (CS) activity per 10⁶ cells.

16. A pharmaceutical composition for increasing levels of lymphoid cells in a subject comprising mitochondrially-enriched target cells and a pharmaceutically acceptable carrier, wherein the mitochondrially-enriched target cells are enriched with exogenous mitochondria.

17. The pharmaceutical composition of claim 16, wherein the mitochondrially-enriched target cells are produced by the method comprising:
(a) obtaining target cells from a subject afflicted with a disease or disorder or a donor;
(b) obtaining exogenous mitochondria from a donor; and
(c) producing mitochondrially-enriched target cells by contacting the target cells with the exogenous mitochondria under conditions allowing the exogenous mitochondria to enter the target cells,
wherein the mitochondrial content of the mitochondrially-enriched target cells is detectably higher than the mitochondrial content of the target cells.

18-30. (canceled)

31. A method for diminishing debilitating effects of a lymphocyte deficiency-related disease or diseases in a subject comprising:
(a) incubating of hematopoietic stem cells (HSCs) with exogenous mitochondria under conditions allowing the exogenous mitochondria to enter the HSCs; and
(b) administering the HSCs from (a) to the subject.

32. (canceled)

33. The method of claim 31, wherein the exogenous mitochondria have undergone at least one freeze-thaw cycle.

34. The method of claim 31, wherein the conditions allowing the exogenous mitochondria to enter the HSCs comprise a ratio of about 0.088-176 mU citrate synthase (CS) activity per 10⁶ cells.

35-62. (canceled)
